SEQUENCE LISTING (12 sequences)

SEQ ID NO: 1 - protein, 452 aa, synthetic construct
EVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCASSKEKATYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 340 aa, synthetic construct
DVQLVESGGGLVQAGGSLRLACAGSGRIFAIYDIAWYRHPPGNQRELVAMIRPVVTEIDYADSVKGRFTISRNNAMKTVYLQMNNLKPEDTAVYYCNAKRPWGSRDEYWGQGTQVTVSSGSGSGQAVVLQEPSLSVSPGGTVTLTCGLSSGSVSTNYYPSWYQQTPGQAPRTLIYYTNTRSSDVPERFSGSIVGNKAALTITGAQPDDESVYFCLLYLGRGIWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 3 - protein, 589 aa, synthetic construct
EVQLVESGGGLVQPGGSLRLSCAASGFTFTSYYISWVRQAPGKGLEWVAEISPYSGSTYYADSVKGRFTISADTSKNTAYLQMNSLRAEDTAVYYCALRARPPIRLHPRGSVMDYWGQGTLVTVSSGGGGSGGGGSGGGGSEVQLVESGGGLVQPGGSLRLSCAASGFTFSHYTLSWVRQAPGKGLEWVSVISGDGSYTYYADSVKGRFTISSDNSKNTLYLQMNSLRAEDTAVYYCARNFIKYVFANWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 335 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQSYTTPPTFGQGTKVEIKGGGGSGGGGSGGGGSDIELTQPPSVSVAPGQTARISCSGDNIGSFYVHWYQQKPGQAPVLVIYDKSNRPSGIPERFSGSNSGNTATLTISGTQAEDEADYYCQSYANTLSLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 5 - protein, 588 aa, synthetic construct
EVQLVESGGGLVQPGGSLRLSCAASGFTFTSYYISWVRQAPGKGLEWVAEISPYSGSTYYADSVKGRFTISADTSKNTAYLQMNSLRAEDTAVYYCALRARPPIRLHPRGSVMDYWGQGTLVTVSSGGGGSGGGGSGGGGSEVQLVESGGGLVQPGGSLRLSCAASGFNISSSYIHWVRQAPGKGLEWVAYIYSSYGSTYYADSVKGRFTISADTSKNTAYLQMNSLRAEDTAVYYCARASWYALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 338 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQSYTTPPTFGQGTKVEIKGGGGSGGGGSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQYWYGVAPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 585 aa, synthetic construct
EVQLVESGGGLVQPGGSLRLSCAASGFTFTSYYISWVRQAPGKGLEWVAEISPYSGSTYYADSVKGRFTISADTSKNTAYLQMNSLRAEDTAVYYCALRARPPIRLHPRGSVMDYWGQGTLVTVSSGGGGSGGGGSGGGGSEVQLVESGGGLVQPGGSLRLSCAASGFNISYSYIHWVRQAPGKGLEWVASIYSSSGSTSYADSVKGRFTISADTSKNTAYLQMNSLRAEDTAVYYCARGAIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 339 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQSYTTPPTFGQGTKVEIKGGGGSGGGGSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQWYSSGHVLITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 589 aa, synthetic construct
EVQLVESGGGLVQPGGSLRLSCAASGFTFTSYYISWVRQAPGKGLEWVAEISPYSGSTYYADSVKGRFTISADTSKNTAYLQMNSLRAEDTAVYYCALRARPPIRLHPRGSVMDYWGQGTLVTVSSGGGGSGGGGSGGGGSEVQLVESGGGLVQPGGSLRLSCAASGFNISYYYIHWVRQAPGKGLEWLASIYPSSGYTYYADSVKGRFTISADTSKNTAYLQMNSLRAEDTAVYYCARSSFYWAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 337 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQSYTTPPTFGQGTKVEIKGGGGSGGGGSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQSYAAYLFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 337 aa, synthetic construct
DVQLVESGGGLVQAGGSLRLACAGSGRIFAIYDIAWYRHPPGNQRELVAMIRPVVTEIDYADSVKGRFTISRNNAMKTVYLQMNNLKPEDTAVYYCNAKRPWGSRDEYWGQGTQVTVSSGSGSGSYVLTQPPSVSVSPGQTASITCSGDKVGHKYASWYQQKPGQSPVLVIYEDSQRPSGIPVRFSGSNSGNTATLTISGTQAMDEADYYCQAWDSSTDVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 12 - protein, 454 aa, synthetic construct
QVQLQQWGAGLLKPSETLSLTCAVSGASFSGHYWTWIRQPPGKGLEWIGEIDHTGSTNYEPSLRSRVTISVDTSKNQFSLNLKSVTAADTAVYYCARGGQGGYDWGHYHGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2024/0391991 A1
(43) Pub. Date: Nov. 28, 2024

(54) MODULATION OF WNT SIGNALLING IN PULMONARY DISORDERS

(71) Applicant: Surrozen Operating, Inc., South San Francisco, CA (US)

(72) Inventors: Russell FLETCHER, Berkeley, CA (US); Kuo-Pao LAI, Campbell, CA (US); Yang LI, Mountain View, CA (US); Wen-Chen YEH, Belmont, CA (US)

(21) Appl. No.: 18/691,419

(22) PCT Filed: Sep. 14, 2022
(86) PCT No.: PCT/US2022/076437

§ 371 Date: Mar. 12, 2024

Related U.S. Application Data
(60) Provisional application No. 63/346,738, filed on May 27, 2022.
(60) Provisional application No. 63/244,071, filed on Sep. 14, 2021.

(51) Int. Cl. C07K 16/22; A61K 39/00; A61P 11/00

(52) U.S. Cl. CPC C07K 16/22; A61P 11/00; A61K 2039/505; C07K 2317/75; C07K 2317/76

(57) ABSTRACT

The present invention provides methods of treating pulmonary disorders with modulators of the Wnt signaling pathway. Also provided are methods of related methods of dosing and pharmaceutical compositions.

### CROSS REFERENCE TO RELATED APPLICATIONS

This application claims priority to U.S. Provisional Application No. 63/244,071, filed on Sep. 14, 2021, and U.S. Provisional Application No. 63/346,738, filed on May 27, 2022, each of which is incorporated by reference herein in its entirety.

### STATEMENT REGARDING SEQUENCE LISTING

The Sequence Listing XML associated with this application is provided in XML file format and is hereby incorporated by reference into the specification. The name of the XML file containing the Sequence Listing XML is SRZN_011_04WO_ST26.xml. The XML file is 16,607 bytes, and created on Sep. 13, 2022, and is being submitted electronically via USPTO Patent Center.

### FIELD OF THE INVENTION

The present invention provides Wnt signal modulators as a treatment for pulmonary disorders, in particular, e.g., pulmonary fibrosis and COPD.

### BACKGROUND OF THE INVENTION

Generation of the alveolus requires intricate interactions between multiple cell lineages to create the complex structure responsible for gas exchange in mammals. Epithelial, mesenchymal, and endothelial cell lineages combine to expand the saccular structure at the distal tips of the branched airways starting around embryonic day 16.5 (E16.5) in mice. Soon thereafter, this rudimentary structure remodels and promotes epithelial and mesenchymal cell communication, which helps integrate the developing vascular network. Remodeling of the alveolus continues postnatally concomitant with specification and maturation of alveolar type 1 (AT1) and type 2 (AT2) epithelial cells until lung maturity is reached at postnatal day 30 (PN30) in mice and into adolescence in humans. Despite the extensive knowledge of earlier stages of lung development including branching morphogenesis, little is known about the cell lineage specific interactions and molecular pathways governing the normal generation of the lung alveolus. Disruption of this process can be deleterious and result in neonatal diseases such as bronchopulmonary dysplasia (BPD), as well as the adult disorders of Idiopathic Pulmonary Fibrosis (IPF) and Chronic Obstructive Pulmonary Disorder (COPD).

Wnt signaling is a critical pathway important for self-renewal and specification of stem cells in multiple organs. Components of the Wnt pathway are expressed in specific patterns during early lung development, and previous work has demonstrated essential roles for Wnt signaling in lung endoderm specification and early development. However, the roles Wnt signaling plays in later stages of lung epithelial differentiation and maturation is still being elucidated. A novel Wnt signaling reporter mouse line (Axin2^{CreERT2-TdTom}), revealed a previously unknown wave of Wnt signaling during alveologenesis (Frank et al., 2016; Nabhan et al., 2018). This cell line defined a sublineage of AT2 epithelial cells called AT2s^{Axin2}, which emerges at the onset of alveologenesis. AT2s^{Axin2} epithelial cells appear to promote lung organoid formation in ex vivo assays and have greater clonal growth potential in vivo during alveologenesis. Importantly, activation of Wnt signaling in the overall AT2 epithelial cell population elicits a similar self-renewal response, promoting enhanced organoid formation, increased proliferation, and increased clonal expansion during alveologenesis. Conversely, inhibition of Wnt signaling in the overall AT2 epithelial cell lineage inhibits organoid formation and AT2 epithelial cell self-renewal and shunts their differentiation towards the ATI epithelial cell lineage. A need exists to balance critical roles for both Wnt agonist and antagonist signaling during lung alveologenesis through expansion of the AT2 epithelial cell population via proliferation, and the subsequent differentiation to AT1 epithelial cell.

In COPD patients, the alveolar epithelium (and hence the gas exchange interface) is significantly reduced, and there is also some bronchiolar fibrosis and inflammation (Barnes et al. 2015). COPD lungs demonstrate a reduction in Wnt/β-catenin signaling components and activity (H A Baarsma and Königshoff 2017; Shi et al. 2017; R. Wang et al. 2011). In mouse models of COPD such as elastase and smoking, the use of small molecule activators of Wnt signaling appeared to restore AT2 cells and alveoli and reduce the amount of airspace enlargement (Kneidinger et al., 2011; Conlon et al., 2020. Therefore, targeted Wnt activation could provide a therapeutic benefit of renewing AT2 cells and restoring alveoli in COPD.

IPF is characterized by the accumulation of fibrotic foci and AT2 epithelial cell hyperplasia (King, Pardo, and Selman 2011). In addition to TGFβ signaling, it is thought that overly active Wnt/β-catenin signaling in mesenchyme/fibroblasts contributes to the overproduction of extracellular matrix and fibrotic foci formation (Chilosi et al. 2003; Shi et al. 2017; H A Baarsma and Königshoff 2017). Recent studies performed at single cell resolution are consistent with this view, demonstrating that overly active Wnt/β-catenin signaling in specific mesenchymal subpopulations can lead to a fibrogenic gene expression program (Zepp et al. 2017). In the alveolar epithelium, there is evidence that the epithelial cells associated with AT2 hyperplasia in IPF demonstrate mixed lineage characteristics of both AT1 and AT2 cells (Xu et al. 2016). Importantly, a subset of AT2 epithelial cells, which express Wnt/β-catenin target gene, Axin-2, are stem/progenitor cells (termed airway epithelial progenitors (AEP)), and AT2/AEP epithelial cell proliferation requires active Wnt signaling (Barkauskas et al. 2013; Desai, Brownfield, and Krasnow 2014; Zacharias et al. 2018; Nabhan et al. 2018). Furthermore, in the adult lung proper and organoid culture studies, ATI epithelial cells are derived from AT2 epithelial cells, which differentiate into ATI cells, and this requires the attenuation of Wnt signaling (Rock et al. 2011; Nabhan et al. 2018; Zacharias et al. 2018). Activation of Wnt signaling in the AT2 cells could provide a therapeutic benefit by promoting their expansion, and then upon their differentiation into ATI cells, lead to restoration of alveoli in lung disease.

Several studies have shown the existence of aberrant basaloid cells and cells that appear to be paused in the transition from AT2 epithelial cell to ATI epithelial cell differentiation and contribute to the production of extracellular matrix in IPF lungs, and these may be overlapping populations (Adams et al., (2020); Strunz et al., (2020); Kobayashi et al., (2020); Haberman et al., (2020)). Furthermore, there is also evidence that modulating Wnt signaling impacts the inflammatory state of macrophages in the lung upon LPS or bleomycin injury (Zhou et al., 2020). An ideal therapeutic approach for pulmonary fibrosis and IPF is to limit myofibroblast-mediated and disease-specific epithelial cell mediated matrix production and promote an anti-inflammatory macrophage phenotype, while promoting AT2/AEP epithelial cell renewal and facilitating AT2 epithelial cell to ATI epithelial cell fate conversion.

A recent scRNA-seq study identified an AT2 transition state (ATO) and SCG3BA2+ terminal respiratory bronchiolar secretory cells, and their analysis found that human and primate AT2 cells are capable of differentiating into ATI cells as well as the terminal respiratory bronchiolar secretory cells via transit through the ATO transition state, demonstrating the multipotency of AT2 cells [Murthy et al., 2022]. In human IPF samples, ATO and SCG3BA2+ terminal respiratory bronichiolar cells were enriched in fibrotic regions [Murthy 2022]. In culture, the depletion of Wnt activation or EGF was necessary for AT2 cells to form ATO cells and terminal bronchiolar secretory cells [Murthy 2022]. Another recent scRNA-seq analysis of distal human lungs also identified a subpopulation of terminal respiratory brionchiolar secretory cells that express SCGB3B2, which they termed respiratory airway secretory (RAS) cells. Their organoid culture studies provided evidence that RAS cells could differentiate into AT2 cells in vitro, and this was robustly enhanced by activating Wnt signaling [Basil et al., 2022]. Together, these findings suggest that activating Wnt signaling in the transition state, disease-associated aberrant epithelial cells, or terminal respiratory bronchiolar secretory cells might be able to direct them to differentiate into AT2 cells and ultimately lead to the restoration of alveoli and limit fibrosis.

Therefore, establishing mechanisms for targeted antagonism and/or agonism of Wnt signaling in specific lung cell populations could provide great therapeutic benefit in fibrotic lung diseases. For example, establishing mechanisms for targeted antagonism and/or agonism of Wnt signaling in disease-specific distal lung epithelial cells (transition state cells, aberrant basaloid cells, or disease-associated terminal bronchiolar respiratory cells) to push them to form AT2 epithelial cells or to differentiate into AT1 epithelial cells could provide a therapeutic benefit of reducing fibrosis in fibrotic lung diseases and COPD. Establishing methods for targeted antagonism and/or agonism of specific fibroblast populations could reduce their pro-fibrogenic activity.

Additionally, establishing mechanisms for targeted Wnt signaling agonism on resident and/or infiltrating immune cells, including macrophages, to promote an anti-inflammatory phenotype and effect could provide a therapeutic benefit for reducing fibrosis and promoting alveolar regeneration in fibrotic lung diseases and COPD.

Furthermore, modulation of Wnt signaling via FZD4 regulation has been suggested to impact alveolar repair in COPD models (Skronska-Wasek et al., (2017). Therefore, establishing methods for targeted agonism or antagonism on endothelial cells, which show enrichment for FZD4 (see, e.g., Adams et al., (2020) supra) could provide a therapeutic benefit for reducing fibrosis in fibrotic lung disease.

The present invention provides compositions and methods to regulate the balance between Wnt signaling agonism and antagonism in a targeted manner.

### SUMMARY OF THE INVENTION

The present invention is based, in part, upon the use of Wnt agonists and antagonists to regulate proliferation of pulmonary AT2 epithelial cells (AT2 cells) and subsequently differentiate these cells into AT1 epithelial cells (AT1 cells), to regenerate healthy lung tissue.

The present invention is based, in part, upon the use of Wnt agonists and antagonists to impact the pathogenic phenotype of disease-specific pulmonary epithelial cells such as AT2/AT1 transition state cells, aberrant basaloid cells, and/or disease-associated SCGB3A2+ respiratory bronchiolar secretory cells and subsequently lead to the renewal of AT2 cells and ATI epithelial cells (AT1 cells), to regenerate healthy lung tissue.

The present invention is based, in part, upon the use of Wnt antagonists and agonists to regulate the response of immune cells in the lung to limit inflammation and provide an environment that is conducive to the proliferation of pulmonary AT2 cells and their subsequent differentiation into AT1 cells to regenerate healthy lung tissue.

In one aspect, the present invention provides a method of treating a subject suffering from a pulmonary disorder comprising administering to the subject an engineered Wnt antagonist and/or an engineered Wnt agonist. In some embodiments, the pulmonary disorder is an interstitial lung disease and can be selected from idiopathic pulmonary fibrosis, cryptogenic organizing pneumonia, desquamative interstitial pneumonitis, nonspecific interstitial pneumonitis, hypersensitivity pneumonitis, acute interstitial pneumonitis, interstitial pneumonia, systemic sclerosis-associated pulmonary fibrosis, sarcoidosis, asbestosis-induced fibrosis, lung injury as the result of acute and chronic lung infections (e.g., viral, bacterial, fungal), pneumonia, aspiration injuries, sepsis, acute respiratory distress syndrome. In other embodiments the pulmonary disorder is chronic obstructive pulmonary disease (COPD), including chronic bronchitis, emphysema, and chronic asthma.

In certain embodiments, the subject is administered the Wnt antagonist alone (i.e., without being administered the Wnt agonist). In particular embodiments, the subject is administered the Wnt antagonist alone, e.g., to treat disorders caused by pulmonary fibrosis. In particular embodiments, the subject is administered the Wnt antagonist alone to treat disorders caused by pulmonary fibrosis where targeting pro-fibrotic cells such as activated myofibroblasts could limit fibrosis.

In certain embodiments, the subject is administered the Wnt agonist alone (i.e., without being administered the Wnt antagonist). In certain embodiments, the subject is administered the Wnt agonist alone, e.g., to treat disorders caused by COPD. In other embodiments, a Wnt agonist alone can be used to treat disorders caused by pulmonary fibrosis.

In certain embodiments, the method comprises administering both the Wnt antagonist and the Wnt agonist. In particular embodiments, the Wnt antagonist and the Wnt agonist are administered sequentially, and in other embodiments the Wnt antagonist and agonist are administered concurrently. In certain embodiments, when administered sequentially, the Wnt antagonist is administered prior to the Wnt agonist. In certain embodiments, when administered sequentially, the Wnt agonist is administered prior to the Wnt antagonist. In certain embodiments, the subject is administered the Wnt antagonist and the Wnt agonist, e.g., to treat an interstitial lung disease, which may be selected from idiopathic pulmonary fibrosis, cryptogenic organizing pneumonia, desquamative interstitial pneumonitis, nonspecific interstitial pneumonitis, hypersensitivity pneumonitis, acute interstitial pneumonitis, interstitial pneumonia, systemic sclerosis-associated pulmonary fibrosis, sarcoidosis, asbestosis-induced fibrosis, lung injury as the result of acute and chronic lung infections (e.g., viral, bacterial, fungal), pneumonia, aspiration injuries, sepsis, and acute respiratory distress syndrome. In other embodiments, the subject is administered the Wnt antagonist and the Wnt agonist, e.g., to treat chronic obstructive pulmonary disease (COPD), including chronic bronchitis, emphysema, and chronic asthma.

In particular embodiments of the methods disclosed herein, the engineered antagonist is selected from the group consisting of: an engineered polypeptide, an engineered antibody containing at least one epitope binding domain, a small molecule, an siRNA, and an antisense nucleic acid molecule. In particular embodiments, the engineered agonist is selected from the group consisting of an engineered polypeptide, an engineered antibody containing at least one epitope binding domain and a small molecule. In a particular embodiment, the Wnt antagonist and Wnt agonist are on one molecule.

In some embodiments, the Wnt antagonist and/or the Wnt agonist incorporates a tissue-targeting molecule. In certain embodiments, the tissue-targeting molecule can be an antibody or fragment thereof that binds to a tissue- or cell-specific cell surface molecule.

### BRIEF DESCRIPTION OF THE DRAWINGS

FIG. 1 shows an illustrative dosing scheme in the bleomycin-induced acute lung injury animal model (Degryse, A. et al. (2010). Bleomycin is dosed at time 0, agonists and/or antagonists are administered six times between week 1 and week 4, and termination and bleed occur following 4 weeks. This animal model is used to test Wnt agonist alone, Wnt antagonist alone, a combination of Wnt agonist and Wnt antagonist, or either or both in combination with anti-fibrotic drug or antagonist alone. The model can be used for three to four weeks.

FIG. 2 shows a dosing scheme in the bleomycin-induced chronic lung injury animal model (Degryse, A. et al. (2010)). Bleomycin is dosed at 0, 2, 4, 6, 8, and 10 weeks, agonists are dosed at 5 and 9 weeks, and termination and bleed occur following 10 weeks. The Wnt antagonist/agonist can be used alone or in combination with one or more anti-fibrotic therapies. This animal model may be used to test Wnt agonist alone, Wnt antagonist alone, a combination of Wnt agonist and Wnt antagonist, or either or both in combination with anti-fibrotic drug therapy.

FIG. 3 shows a dosing scheme for the cigarette smoke induced emphysema animal model (Baarsma, H. et al. (2017)). Agonists are dosed four times between day 30 and day 40 following initiation of cigarette smoking, and termination and bleed occur following 42 days.

FIG. 4 shows a dosing scheme for the elastase induced emphysema animal model (Baarsma, H. et al. (2017)). Elastase is dosed at time 0, agonists are dosed four times between day 8 and day 14 following initiation of elastase treatment, and termination and bleed occur following 21 days.

FIGS. 5A and 5B show the ability of a Surrozen Wnt Activating Protein (SWAP), SWAP1 in Table 1, to promote AT2 proliferation and alveolar organoid growth in in vitro assays. The two top images of FIG. 5A are micrographs of AT2 organoids at different magnification. The two bottom images of FIG. 5A show expression of the human AT2 cell marker, HTII-280, in AT2 organoids and KI67 expression assessed by immunofluorescence.

FIG. 5B is a graph showing cell viability on control cells and cells treated with RSPO1, SWAP1+RSPO1, or CHIR99021. AT2 organoids express the human AT2 cell marker, HTII-280, and proliferate in culture (KI67+ cells). The FZD1, 2, 5, 7, 8-specific Wnt Signaling Agonist Protein (SWAP), SWAP1, in combination with RSPO1 expands the AT2 organoid cultures as assessed by CellTiter-Glo assay, a measure of cell viability, reflective of culture growth. The SWAP1/RSPO1 is more effective than the small molecule, CHIR99021, an activator of Wnt signaling.

FIG. 6 shows the ability of multi-FZD-specific and mono-FZD-specific Surrozen Wnt Signaling Agonist Proteins (SWAPs) to expand AT2 organoids, including the ability of mono-FZD4- and mono-FZD5-specific SWAPs to have this impact. The graph shows organoid diameter of control treated organoid, or organoid treated with CHIR99021, FZD1,2,7-specific SWAP2, FZD5,8-specific SWAP3, or FZD4-mono-specific SWAP4.

FIG. 7 shows the ability of Wnt signaling activation via a FZD1,2,5,7,8-specific SWAP5/RSPO combination to reduce the area of the lung that is fibrotic and to lower the severity of fibrosis in the acute bleomycin mouse model. The diagram shows the dosing scheme in the bleomycin induced animal acute lung injury model. Bleomycin was dosed at time 0, saline, anti-GFP antibody, or a combination of FZD multi-Fzd-specific SWAP5+RSPO was dosed four times between week 1 and week 3, and termination and bleed occurred following three weeks. The left graph shows percent of lung affected by fibrosis following the indicated treatments, and the right graph shows the fibrosis score following the indicated treatments. A reduction in the extent of fibrosis in the acute bleomycin model was seen using a SWAP5/RSPO combination (Combo).

FIGS. 8A and 8B show the ability of SWAP2 and SWAP3, which are specific to FZD1,2,7 and FZD5,8, respectively, to induce human AT2 cell organoid expansion alone (in the absence of RSPO) in a dose-dependent manner. SWAP treatment alone induced more AT2 organoid expansion than the small molecule pathway activator, Chir99021 (FIG. 8A). Organoid expansion was assessed using the CellTiter-Glo assay (FIG. 8B).

FIG. 9 shows that a combination of SWAP and RSPO2 together expands mouse AT2 cell organoids to the level seen with the small molecule activator, Chir99021. Organoid expansion was assessed with the CellTiter-Glo assay.

FIGS. 10A-10D show the ability of Wnt signaling activation via a FZD1,2,5,7,8-specific SWAP5 alone and RSPO2 alone to reduce the area of the lung that is fibrotic and to lower the severity of fibrosis in the acute bleomycin lung injury mouse model. FIG. 10A shows the dosing scheme in the bleomycin-induced acute lung injury animal model. Bleomycin was dosed at time 0, and saline, anti-GFP antibody, the multi-Fzd-specific SWAP5, or RSPO2 alone at one of two different doses was dosed four times between week 1 and week 3, and termination and bleed occurred following three weeks. FIG. 10B shows the Modified Ashcroft fibrosis score following the indicated treatments. FIG. 10C shows percent of lung affected by fibrosis following the indicated treatments. FIG. 10D shows the percentage of total smooth muscle cx-actin (ACTA2) expression in the lung (ACTA2 expression is correlated with myofibroblasts that secrete the extracellular matrix contributing to fibrosis, see, e.g., Mitchell et al (1989) Lab Invest. 60:643-650). A reduction in the extent of fibrosis in the acute bleomycin model was seen using SWAP5 alone and RSPO2 alone. Statistical comparisons are made to the anti-GFP treatment condition.

FIGS. 11A-11H show the ability of Wnt signaling activation via a FZD5,8-specific SWAP (SWAP3) alone to reduce the area of the lung that is fibrotic and to lower the severity of fibrosis (Modified Ashcroft Score) in the acute bleomycin mouse model. FIG. 11A shows a schematic of the dosing in the bleomycin-induced acute lung injury animal model. Bleomycin was dosed at time 0, and anti-GFP antibody, the multi-Fzd-specific SWAP5, or the Fzd5,8-specific SWAP alone at one of three different doses was dosed four times between week 1 and week 3, and termination and bleed occurred following three weeks. FIG. 11B shows the Modified Ashcroft fibrosis score following the indicated treatments. FIG. 11C shows percent of lung affected by fibrosis following the indicated treatments. A reduction in the extent of fibrosis in the acute bleomycin model was also seen using SWAP3 alone. FIG. 11D shows a reduction of fibrosis by SWAP3 as measured by a percent of ACTA2, in a bleomycin injury mouse model. FIG. 11E highlights the reduction in inflammation that accompanies the reduction in fibrosis based on histopathology assessment of hematoxylin and eosin-stained tissue sections by H&E based pathology scores. FIGS. 11F-11H show the reduction in cytokines (IL6, IL1B, GRO/CXCL1, respectively) in the lung tissue of the acute bleomycin mouse fibrosis model as assessed by the Meso Scale Discovery platform. Quantification included all animals treated, including those terminated early due to body weight decrease. Statistical comparisons are made to the anti-GFP treatment condition.

FIGS. 12A-12B show the ability of Wnt signaling activation via a FZD1,2,7-specific SWAP (SWAP6) alone to reduce the area of the lung that is fibrotic and to lower the severity of fibrosis (Modified Ashcroft Score) in the acute bleomycin mouse model. FIG. 12A shows the dosing scheme in the bleomycin-induced acute lung injury animal model. Bleomycin was dosed at time 0, and anti-GFP antibody, the multi-Fzd-specific SWAP5, or the Fzd1,2,7-specific SWAP6 alone at one of three different doses was dosed four times between week 1 and week 3, and termination and bleed occurred following three weeks. FIG. 12B shows the Modified Ashcroft fibrosis score following the indicated treatments. SWAP6 caused a trend of reduction in the extent of fibrosis in the acute bleomycin model. Quantification included all animals treated, including those terminated early due to body weight decrease.

### DETAILED DESCRIPTION

As used herein, including the appended claims, the singular forms of words such as “a,” “an,” and “the,” include their corresponding plural references unless the context clearly dictates otherwise.

All references cited herein are incorporated by reference to the same extent as if each individual publication, patent application, or patent, was specifically and individually indicated to be incorporated by reference.

### I. Definitions

“Activity” of a molecule may describe or refer to the binding of the molecule to a ligand or to a receptor, to catalytic activity, to the ability to stimulate gene expression, to antigenic activity, to the modulation of activities of other molecules, and the like. “Activity” of a molecule may also refer to activity in modulating or maintaining cell-to-cell interactions, e.g., adhesion, or activity in maintaining a structure of a cell, e.g., cell membranes or cytoskeleton. “Activity” may also mean specific activity, e.g., [catalytic activity]/[mg protein], or [immunological activity]/[mg protein], or the like.

The terms “administering” or “introducing” or “providing”, as used herein, refer to delivery of a composition to a cell, to cells, tissues and/or organs of a subject, or to a subject. Such administering or introducing may take place in vivo, in vitro or ex vivo.

As used herein, the term “antibody” means an isolated or recombinant binding agent that comprises the necessary variable region sequences to specifically bind an antigenic epitope. Therefore, an antibody is any form of antibody or fragment thereof that exhibits the desired biological activity, e.g., binding the specific target antigen. Thus, it is used in the broadest sense and specifically covers monoclonal antibodies (including full-length monoclonal antibodies), polyclonal antibodies, human antibodies, humanized antibodies, chimeric antibodies, nanobodies, diabodies, multispecific antibodies (e.g., bispecific antibodies), and antibody fragments including but not limited to scFv, Fab, and Fab2, so long as they exhibit the desired biological activity. Antibodies further comprise fusion polypeptides and related molecules that comprise an antibody or fragment thereof.

“Antibody fragments” comprise a portion of an intact antibody, for example, the antigen-binding or variable region of the intact antibody. Examples of antibody fragments include Fab, Fab′, F(ab′) 2, and Fv fragments; diabodies; linear antibodies (e.g., Zapata et al., Protein Eng. 8 (10): 1057-1062 (1995)); single-chain antibody molecules (e.g., scFv); and multispecific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′) 2 fragment that has two antigen combining sites and is still capable of cross-linking antigen. Antibody fragments include functional fragment that bind the same antigen as the intact antibody.

The term “antigen” refers to a molecule or a portion of a molecule capable of being bound by a selective binding agent, such as an antibody, and 30 additionally capable of being used in an animal to produce antibodies capable of binding to an epitope of that antigen. In certain embodiments, a binding agent (e.g., a Wnt surrogate molecule or binding region thereof, or a Wnt antagonist) is said to specifically bind an antigen when it preferentially recognizes its target antigen in a complex mixture of proteins and/or macromolecules.

The term “antigen-binding fragment” as used herein refers to a polypeptide fragment that contains at least one CDR of an immunoglobulin heavy and/or light chain, or of a Nanobody® (Nab), that binds to the antigen of interest, in particular to one or more Fzd receptors, or to LRP5 and/or LRP6. In this regard, an antigen-binding fragment of the herein described antibodies may comprise 1, 2, 3, 4, 5, or all 6 CDRs of a VH and VL from antibodies that bind one or more Fzd receptors or LRP5 and/or LRP6.

As used herein, the terms “biological activity” and “biologically active” refer to the activity attributed to a particular biological element in a cell. For example, the “biological activity” of an Wnt agonist, or fragment or variant thereof refers to the ability to mimic or enhance Wnt signals. As another example, the biological activity of a polypeptide or functional fragment or variant thereof refers to the ability of the polypeptide or functional fragment or variant thereof to carry out its native functions of, e.g., binding, enzymatic activity, etc. As a third example, the biological activity of a gene regulatory element, e.g. promoter, enhancer, Kozak sequence, and the like, refers to the ability of the regulatory element or functional fragment or variant thereof to regulate, i.e. promote, enhance, or activate the translation of, respectively, the expression of the gene to which it is operably linked.

The term “bifunctional antibody,” as used herein, refers to an antibody that comprises a first arm having a specificity for one antigenic site and a second arm having a specificity for a different antigenic site, i.e., the bifunctional antibodies have a dual specificity.

“Bispecific antibody” is used herein to refer to a full-length antibody that is generated by quadroma technology (see Milstein et al., Nature, 305 (5934): 537-540 (1983)), by chemical conjugation of two different monoclonal antibodies (see, Staerz et al., Nature, 314 (6012): 628-631 (1985)), or by knob-into-hole or similar approaches, which introduce mutations in the Fc region (see Holliger et al., Proc. Natl. Acad. Sci. USA, 90 (14): 6444-6448 (1993)), resulting in multiple different immunoglobulin species of which only one is the functional bispecific antibody. A bispecific antibody binds one antigen (or epitope) on one of its two binding arms (one pair of HC/LC), and binds a different antigen (or epitope) on its second arm (a different pair of HC/LC). By this definition, a bispecific antibody has two distinct antigen-binding arms (in both specificity and CDR sequences), and is monovalent for each antigen to which it binds.

By “comprising,” it is meant that the recited elements are required in, for example, the composition, method, kit, etc., but other elements may be included to form the, for example, composition, method, kit etc. within the scope of the claim. For example, an expression cassette “comprising” a gene encoding a therapeutic polypeptide operably linked to a promoter is an expression cassette that may include other elements in addition to the gene and promoter, e.g., poly-adenylation sequence, enhancer elements, other genes, linker domains, etc.

By “consisting essentially of,” it is meant a limitation of the scope of the, for example, composition, method, kit, etc., described to the specified materials or steps that do not materially affect the basic and novel characteristic(s) of the, for example, composition, method, kit, etc. For example, an expression cassette “consisting essentially of” a gene encoding a therapeutic polypeptide operably linked to a promoter and a polyadenylation sequence may include additional sequences, e.g., linker sequences, so long as they do not materially affect the transcription or translation of the gene. As another example, a variant, or mutant, polypeptide fragment “consisting essentially of” a recited sequence has the amino acid sequence of the recited sequence plus or minus about 10 amino acid residues at the boundaries of the sequence based upon the full length naïve polypeptide from which it was derived, e.g. 10, 9, 8, 7, 6, 5, 4, 3, 2 or 1 residue less than the recited bounding amino acid residue, or 1, 2, 3, 4, 5, 6, 7, 8, 9, or 10 residues more than the recited bounding amino acid residue.

By “consisting of,” it is meant the exclusion from the composition, method, or kit of any element, step, or ingredient not specified in the claim. For example, a polypeptide or polypeptide domain “consisting of” a recited sequence contains only the recited sequence.

A “control element” or “control sequence” is a nucleotide sequence involved in an interaction of molecules that contributes to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters and enhancers. A promoter is a DNA region capable under certain conditions of binding RNA polymerase and initiating transcription of a coding region usually located downstream (in the 3′ direction) from the promoter.

An “epitope” is specific region on an antigen that an antibody recognizes and binds to, and is also referred to as the “antigenic determinant”. An epitope is usually 5-8 amino acids long on the surface of the protein. Proteins are three dimensionally folded structures, and an epitope may only be recognized in its form as it exists in solution, or its native form. When an epitope is made up of amino acids that are brought together by the three-dimensional structure, the epitope is conformational, or discontinuous. If the epitope exists on a single polypeptide chain, it is a continuous, or linear epitope. Depending on the epitope an antibody recognizes, it may bind only fragments or denatured segments of a protein, or it may also be able to bind the native protein.

The portion of an antibody or antibody fragment thereof that recognizes an epitope is referred to as the “epitope binding domain” or “antigen binding domain”. The epitope or antigen binding domain of an antibody or antibody fragment is in the Fab fragment and the effector functions in the Fc fragment. Six segments, known as complementarity determining regions (CDRs) within the variable regions (VH and VL) of the heavy and light chains loop out from the framework (FR regions) globular structure of the rest of the antibody and interact to form an exposed surface at one end of the molecule. This is the antigen binding domain. Generally, 4-6 of the CDRs will be directly involved in binding antigen, although fewer can provide the main binding motifs.

An “expression vector” is a vector, e.g., plasmid, mini-circle, viral vector, liposome, and the like as discussed herein or as known in the art, comprising a region which encodes a gene product of interest, and is used for effecting the expression of the gene product in an intended target cell. An expression vector also comprises control elements, e.g., promoters, enhancers, UTRs, miRNA targeting sequences, etc., operatively linked to the encoding region to facilitate expression of the gene product in the target. The combination of control elements and a gene or genes to which they are operably linked for expression is sometimes referred to as an “expression cassette,” a large number of which are known and available in the art or can be readily constructed from components that are available in the art.

As used herein, the term “FR set” refers to the four flanking amino acid sequences which frame the CDRs of a CDR set of a heavy or light chain V region. Some FR residues may contact bound antigen; however, FRs are primarily responsible for folding the V region into the antigen-binding site, particularly the FR residues directly adjacent to the CDRs. Within FRs, certain amino residues and certain structural features are very highly conserved. In this regard, all V region sequences contain an internal disulfide loop of around 90 amino acid residues. When the V regions fold into a binding-site, the CDRs are displayed as projecting loop motifs which form an antigen-binding surface. It is generally recognized that there are conserved structural regions of FRs which influence the folded shape of the CDR loops into certain “canonical” structures-regardless of the precise CDR amino acid sequence. Further, certain FR residues are known to participate in non-covalent interdomain contacts which stabilize the interaction of the antibody heavy and light chains.

The terms “individual,” “host,” “subject,” and “patient” are used interchangeably herein, and refer to a mammal, including, but not limited to, human and non-human primates, including simians and humans; mammalian sport animals (e.g., horses); mammalian farm animals (e.g., sheep, goats, etc.); mammalian pets (dogs, cats, etc.); and rodents (e.g., mice, rats, etc.).

A “monoclonal antibody” refers to a homogeneous antibody population wherein the monoclonal antibody is comprised of amino acids (naturally occurring and non-naturally occurring) that are involved in the selective binding of an epitope. Monoclonal antibodies are highly specific, being directed against a single epitope. The term “monoclonal antibody” encompasses not only intact monoclonal antibodies and full-length monoclonal antibodies, but also fragments thereof (such as Fab, Fab′, F(ab′) 2, Fv), single chain (scFv), Nanobodies®, variants thereof, fusion proteins comprising an antigen-binding fragment of a monoclonal antibody, humanized monoclonal antibodies, chimeric monoclonal antibodies, and any other modified configuration of the immunoglobulin molecule that comprises an antigen-binding fragment (epitope recognition site) of the required specificity and the ability to bind to an epitope, including Wnt surrogate molecules disclosed herein. It is not intended to be limited as regards the source of the antibody or the manner in which it is made (e.g., by hybridoma, phage selection, recombinant expression, transgenic animals, etc.). The term includes whole immunoglobulins as well as the fragments etc. described above under the definition of “antibody”.

The term “native” or “wild-type” as used herein refers to a nucleotide sequence, e.g., gene, or gene product, e.g., RNA or protein, that is present in a wild-type cell, tissue, organ or organism. The term “variant” as used herein refers to a mutant of a reference polynucleotide or polypeptide sequence, for example a native polynucleotide or polypeptide sequence, i.e., having less than 100% sequence identity with the reference polynucleotide or polypeptide sequence. Put another way, a variant comprises at least one amino acid difference (e.g., amino acid substitution, amino acid insertion, amino acid deletion) relative to a reference polynucleotide sequence, e.g., a native polynucleotide or polypeptide sequence. For example, a variant may be a polynucleotide having a sequence identity of 50% or more, 60% or more, or 70% or more with a full-length native polynucleotide sequence, e.g., an identity of 75% or 80% or more, such as 85%, 90%, or 95% or more, for example, 98% or 99% identity with the full-length native polynucleotide sequence. As another example, a variant may be a polypeptide having a sequence identity of 70% or more with a full-length native polypeptide sequence, e.g., an identity of 75% or 80% or more, such as 85%, 90%, or 95% or more, for example, 98% or 99% identity with the full-length native polypeptide sequence. Variants may also include variant fragments of a reference, e.g., native, sequence sharing a sequence identity of 70% or more with a fragment of the reference, e.g., native, sequence, e.g., an identity of 75% or 80% or more, such as 85%, 90%, or 95% or more, for example, 98% or 99% identity with the native sequence.

“Operatively linked” or “operably linked” refers to a juxtaposition of genetic elements, wherein the elements are in a relationship permitting them to operate in the expected manner. For instance, a promoter is operatively linked to a coding region if the promoter helps initiate transcription of the coding sequence. There may be intervening residues between the promoter and coding region so long as this functional relationship is maintained.

As used herein, the terms “polypeptide,” “peptide,” and “protein” refer to polymers of amino acids of any length. The terms also encompass an amino acid polymer that has been modified; for example, to include disulfide bond formation, glycosylation, lipidation, phosphorylation, or conjugation with a labeling component.

The term “polynucleotide” refers to a polymeric form of nucleotides of any length, including deoxyribonucleotides or ribonucleotides, or analogs thereof. A polynucleotide may comprise modified nucleotides, such as methylated nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the invention described herein that is a polynucleotide encompasses both the double-stranded form and each of two complementary single-stranded forms known or predicted to make up the double-stranded form.

A polynucleotide or polypeptide has a certain percent “sequence identity” to another polynucleotide or polypeptide, meaning that, when aligned, that percentage of bases or amino acids are the same when comparing the two sequences. Sequence similarity can be determined in a number of different manners. To determine sequence identity, sequences can be aligned using the methods and computer programs, including BLAST, available over the worldwide web at ncbi.nlm.nih.gov/BLAST/. Unless indicated to the contrary, sequence identity is determined using BLAST. Another alignment algorithm is FASTA, available in the Genetics Computing Group (GCG) package, from Madison, Wis., USA, a wholly owned subsidiary of Oxford Molecular Group, Inc. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Of particular interest are alignment programs that permit gaps in the sequence. The Smith-Waterman is one type of algorithm that permits gaps in sequence alignments. See Meth. Mol. Biol. 70:173-187 (1997). Also, the GAP program using the Needleman and Wunsch alignment method can be utilized to align sequences. See J. Mol. Biol. 48:443-453 (1970)

Of interest is the BestFit program using the local homology algorithm of Smith and Waterman (Advances in Applied Mathematics 2:482-489 (1981) to determine sequence identity. The gap generation penalty will generally range from 1 to 5, usually 2 to 4 and in many embodiments will be 3. The gap extension penalty will generally range from about 0.01 to 0.20 and in many instances will be 0.10. The program has default parameters determined by the sequences inputted to be compared. Preferably, the sequence identity is determined using the default parameters determined by the program. This program is available also from Genetics Computing Group (GCG) package, from Madison, Wis., USA.

Another program of interest is the FastDB algorithm. FastDB is described in Current Methods in Sequence Comparison and Analysis, Macromolecule Sequencing and Synthesis, Selected Methods and Applications, pp. 127-149, 1988, Alan R. Liss, Inc. Percent sequence identity is calculated by FastDB based upon the following parameters: Mismatch Penalty: 1.00; Gap Penalty: 1.00; Gap Size Penalty: 0.33; and Joining Penalty: 30.0.

A “promoter” as used herein encompasses a DNA sequence that directs the binding of RNA polymerase and thereby promotes RNA synthesis, i.e., a minimal sequence sufficient to direct transcription. Promoters and corresponding protein or polypeptide expression may be ubiquitous, meaning strongly active in a wide range of cells, tissues and species or cell-type specific, tissue-specific, or species specific. Promoters may be “constitutive,” meaning continually active, or “inducible,” meaning the promoter can be activated or deactivated by the presence or absence of biotic or abiotic factors. Also included in the nucleic acid constructs or vectors of the invention are enhancer sequences that may or may not be contiguous with the promoter sequence. Enhancer sequences influence promoter-dependent gene expression and may be located in the 5′ or 3′ regions of the native gene.

“Recombinant,” as applied to a polynucleotide means that the polynucleotide is the product of various combinations of cloning, restriction or ligation steps, and other procedures that result in a construct that is distinct from a polynucleotide found in nature.

The terms “treatment”, “treating” and the like are used herein to generally mean obtaining a desired pharmacologic and/or physiologic effect. The effect may be therapeutic in terms of a partial or complete cure for a disease and/or adverse effect attributable to the disease. “Treatment” as used herein covers any treatment of a disease in a mammal, and includes: (a) inhibiting the disease, i.e., arresting its development; or (b) relieving the disease, i.e., causing regression of the disease. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease. The term “prevent” means completely or partially preventing or inhibiting a disease or symptom thereof, e.g., reducing the likelihood that the disease or symptom thereof occurs in the subject.

The practice of the present invention will employ, unless otherwise indicated, conventional techniques of cell biology, molecular biology techniques), microbiology, biochemistry and immunology, which are within the scope of those of skill in the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology” (D. M. Weir & C. C.

Blackwell, eds.); “Gene Transfer Vectors for Mammalian Cells” (J. M. Miller & M. P. Calos, eds., 1987); “Current Protocols in Molecular Biology” (F. M. Ausubel et al., eds., 1987); “PCR: The Polymerase Chain Reaction”, (Mullis et al., eds., 1994); and “Current Protocols in Immunology” (J. E. Coligan et al., eds., 1991), each of which is expressly incorporated by reference herein.

Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details or with other methods. The present invention is not limited by the illustrated ordering of acts or events, as some acts may occur in different orders and/or concurrently with other acts or events. Furthermore, not all illustrated acts or events are required to implement a methodology in accordance with the present invention.

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

All publications mentioned herein are incorporated herein by reference to disclose and describe the methods and/or materials in connection with which the publications are cited. It is understood that the present disclosure supersedes any disclosure of an incorporated publication to the extent there is a contradiction.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

Unless otherwise indicated, all terms used herein have the same meaning as they would to one skilled in the art and the practice of the present invention will employ, conventional techniques of microbiology and recombinant DNA technology, which are within the knowledge of those of skill of the art.

### II. General

The present invention provides methods of modulating Wnt signals to ameliorate pulmonary disorders, including but not limited to, chronic obstructive pulmonary disorder (COPD) and idiopathic pulmonary fibrosis. In particular embodiments, the present invention provides a Wnt/β-catenin signaling antagonist for myofibroblast, immune cells, pulmonary specific AT2 cells, terminal respiratory bronchiolar cells, and/or aberrant epithelial cells, and a Wnt/β-catenin agonist, to promote the self-renewal and/or differentiation of these cells in order to alleviate various pulmonary disorders.

Wnt (“Wingless-related integration site” or “Wingless and Int-1” or “Wingless-Int”) ligands and their signals play key roles in the control of development, homeostasis and regeneration of many essential organs and tissues, including bone, liver, skin, stomach, intestine, kidney, central nervous system, mammary gland, taste bud, ovary, cochlea, lung, and many other tissues (reviewed, e.g., by Clevers, Loh, and Nusse, 2014; 346:1248012). Modulation of Wnt signaling pathways has potential for treatment of degenerative diseases and tissue injuries.

One of the challenges for modulating Wnt signaling as a therapeutic is the existence of multiple Wnt ligands and Wnt receptors, Frizzled 1-10 (Fzd1-10), with many tissues expressing multiple and overlapping Fzds. Canonical Wnt signals also involve Low-density lipoprotein (LDL) receptor-related protein 5 (LRP5) or Low-density lipoprotein (LDL) receptor-related protein 6 (LRP6) as co-receptors, which are broadly expressed in various tissues, in addition to Fzds.

R-spondins 1-4 are a family of ligands that amplify Wnt signals. Each of the R-spondins work through a receptor complex that contains Zinc and Ring Finger 3 (ZNRF3) or Ring Finger Protein 43 (RNF43) on one end and a Leucine-rich repeat-containing G-protein coupled receptor 4-6 (LGR4-6) on the other (reviewed, e.g., by Knight and Hankenson 2014, Matrix Biology; 37:157-161). R-spondins might also work through additional mechanisms of action. ZNRF3 and RNF43 are two membrane-bound E3 ligases specifically targeting Wnt receptors (Fzd1-10 and LRP5 or LRP6) for degradation. Binding of an R-spondin to ZNRF3/RNF43 and LGR4-6 causes clearance or sequestration of the ternary complex, which removes E3 ligases from Wnt receptors and stabilizes Wnt receptors, resulting in enhanced Wnt signals. Each R-spondin contains two Furin domains (1 and 2), with Furin domain 1 binding to ZNRF3/RNF43, and Furin domain 2 binding to LGR4-6. Fragments of R-spondins containing Furin domains 1 and 2 are sufficient for amplifying Wnt signaling. While R-spondin effects depend on Wnt signals, since both LGR4-6 and ZNRF3/RNF43 are widely expressed in various tissues, the effects of R-spondins are not tissue-specific.

In some embodiments, the Wnt/β-catenin signaling antagonist or agonist can include binding agents or epitope binding domains that bind one or more Fzd receptors and inhibit or enhance Wnt signaling. In certain embodiments, the agent or antibody specifically binds to the cysteine-rich domain (CRD) within the human frizzled receptor(s) to which it binds. Additionally, antagonistic binding agents containing epitope-binding domains against LRP can also be used. In some embodiments, the Wnt/β-catenin antagonist possesses binding agents or epitope binding domains that bind E3 ligases ZNRF3/RNF43 and one or more FZD receptors or one or more LRP co-receptors to promote the degradation of FZD or LRP receptors, and this molecule can also contain a binding domain that binds a cell type specific epitope for targeting. The E3 ligase agonist antibodies or fragments thereof can be single molecules or combined with other Wnt antagonists, e.g., Fzd receptor antagonists, LRP receptor antagonists, etc.

As is well known in the art, an antibody is an immunoglobulin molecule capable of specific binding to a target such as a carbohydrate, polynucleotide, lipid, polypeptide, etc., through at least on epitope binding domain, located on the variable region of the immunoglobulin molecule. As used herein, the term encompasses not only intact polyclonal or monoclonal antibodies, but also fragments thereof containing epitope binding domains (e.g., dAb, Fab, Fab′, (F(ab′) 2, Fv, single chain (scFv), Nanobodies® (Nabs), DVD-Igs, synthetic variants thereof, naturally occurring variants, fusion proteins comprising and epitope binding domain, humanized antibodies, chimeric antibodies, and any other modified configuration of the immunoglobulin molecule that comprises an antigen-binding site or fragment (epitope recognition site) of the required specificity. “Diabodies,” multivalent or multispecific fragments constructed by gene fusion (WO94/13804; P. Holliger et al., Proc. Natl. Acad. Sci. USA 90 6444-6448, 1993) are also a particular form of antibody contemplated herein. Minibodies comprising a scFv joined to a CH3 domain are also included herein (S. Hu et al., Cancer Res., 56, 3055-3061, 1996). See e.g., Ward, E. S. et al., Nature 341, 544-546 (1989); Bird et al., Science, 242, 423-426, 1988; Huston et al., PNAS USA, 85, 5879-5883, 1988); PCT/US92/09965; WO94/13804; P. Holliger et al., Proc. Natl. Acad. Sci. USA 90 6444-6448, 1993; Y. Reiter et al., Nature Biotech, 14, 1239-1245, 1996; S. Hu et al., Cancer Res., 56, 3055-3061, 1996.

The proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab′) 2 fragment which comprises both antigen-binding sites. An Fv fragment for use according to certain embodiments of the present disclosure can be produced by preferential proteolytic cleavage of an IgM, and on rare occasions of an IgG or IgA immunoglobulin molecule. Fv fragments are, however, more commonly derived using recombinant techniques known in the art. The Fv fragment includes a non-covalent VH::VL heterodimer including an antigen-binding site which retains much of the antigen recognition and binding capabilities of the native antibody molecule. Inbar et al. (1972) Proc. Nat. Acad. Sci. USA 69:2659-2662; Hochman et al. (1976) Biochem 15:2706-2710; and Ehrlich et al. (1980) Biochem 19:4091-4096.

In certain embodiments, single chain Fv or scFv antibodies are contemplated. For example, Kappa bodies (Ill et al., Prot. Eng. 10:949-57 (1997)); minibodies (Martin et al., EMBO J 13:5305-9 (1994)); diabodies (Holliger et al., PNAS 90:6444-8 (1993)); or Janusins (Traunecker et al., EMBO J 10:3655-59 (1991) and Traunecker et al., Int. J. Cancer Suppl. 7:51-52 (1992)), may be prepared using standard molecular biology techniques following the teachings of the present application with regard to selecting antibodies having the desired specificity. In still other embodiments, bispecific or chimeric antibodies may be made that encompass the ligands of the present disclosure. For example, a chimeric antibody may comprise CDRs and framework regions from different antibodies, while bispecific antibodies may be generated that bind specifically to one or more Fzd receptors through one binding domain and to a second molecule through a second binding domain. These antibodies may be produced through recombinant molecular biological techniques or may be physically conjugated together.

A scFv polypeptide is a covalently linked VH::VL heterodimer which is expressed from a gene fusion including VH- and VL-encoding genes linked by a peptide-encoding linker (Huston et al. (1988) Proc. Nat. Acad. Sci. USA 85 (16): 5879-5883). A number of methods have been described to discern chemical structures for converting the naturally aggregated-but chemically separated-light and heavy polypeptide chains from an antibody V region into an scFv molecule which will fold into a three-dimensional structure substantially similar to the structure of an antigen-binding site. See, e.g., U.S. Pat. Nos. 5,091,513 and 5,132,405, to Huston et al.; and U.S. Pat. No. 4,946,778, to Ladner et al.

In certain embodiments, an antibody as described herein is in the form of a diabody. Diabodies (dAb) are multimers of polypeptides, each polypeptide comprising a first domain comprising a binding region of an immunoglobulin light chain and a second domain comprising a binding region of an immunoglobulin heavy chain, the two domains being linked (e.g., by a peptide linker) but unable to associate with each other to form an antigen binding site: antigen binding sites are formed by the association of the first domain of one polypeptide within the multimer with the second domain of another polypeptide within the multimer (WO94/13804).

A dAb fragment of an antibody consists of a VH domain (Ward, E. S. et al., Nature 341, 544-546 (1989)).

Where bispecific antibodies are to be used, these may be conventional bispecific antibodies, which can be manufactured in a variety of ways (Holliger, P. and Winter G., Current Opinion Biotechnol. 4, 446-449 (1993)), e.g., prepared chemically or from hybrid hybridomas, or may be any of the bispecific antibody fragments mentioned above. Diabodies and scFvs can be constructed without an Fc region, using only variable domains, potentially reducing the effects of anti-idiotypic reaction.

Bispecific diabodies, as opposed to bispecific whole antibodies, may also be particularly useful because they can be readily constructed and expressed in E. coli. Diabodies (and many other polypeptides such as antibody fragments) of appropriate binding specificities can be readily selected using phage display (WO94/13804) from libraries. If one arm of the diabody is to be kept constant, for instance, with a specificity directed against antigen X, then a library can be made where the other arm is varied and an antibody of appropriate specificity selected. Bispecific whole antibodies may be made by knobs-into-holes engineering (J. B. B. Ridgeway et al., Protein Eng., 9, 616-621 (1996)).

In certain embodiments, the antibodies described herein may be provided in the form of a UniBody®. A UniBody® is an IgG4 antibody with the hinge region removed (see GenMab Utrecht, The Netherlands; see also, e.g., US20090226421). This proprietary antibody technology creates a stable, smaller antibody format with an anticipated longer therapeutic window than current small antibody formats. IgG4 antibodies are considered inert and thus do not interact with the immune system. Fully human IgG4 antibodies may be modified by eliminating the hinge region of the antibody to obtain half-molecule fragments having distinct stability properties relative to the corresponding intact IgG4 (GenMab, Utrecht). Halving the IgG4 molecule leaves only one area on the UniBody® that can bind to cognate antigens (e.g., disease targets) and the UniBody® therefore binds univalently to only one site on target cells.

In certain embodiments, antibodies and antigen-binding fragments thereof as described herein include a heavy chain and a light chain CDR set, respectively interposed between a heavy chain and a light chain framework region (FR) set, which provide support to the CDRs and define the spatial relationship of the CDRs relative to each other. As used herein, the term “CDR set” refers to the three hypervariable regions of a heavy or light chain V region. Proceeding from the N-terminus of a heavy or light chain, these regions are denoted as “CDR1,” “CDR2,” and “CDR3” respectively. An antigen-binding site, therefore, includes six CDRs, comprising the CDR set from each of a heavy and a light chain V region. A polypeptide comprising a single CDR, (e.g., a CDR1, CDR2 or CDR3) is referred to herein as a “molecular recognition unit. “Crystallographic analysis of a number of antigen-antibody complexes has demonstrated that the amino acid residues of CDRs form extensive contact with bound antigen, wherein the most extensive antigen contact is with the heavy chain CDR3. Thus, the molecular recognition units are primarily responsible for the specificity of an antigen-binding site.

As used herein, the term “FR set” refers to the four flanking amino acid sequences which frame the CDRs of a CDR set of a heavy or light chain V region. Some FR residues may contact bound antigen; however, FRs are primarily responsible for folding the V region into the antigen-binding site, particularly the FR residues directly adjacent to the CDRs. Within FRs, certain amino residues and certain structural features are very highly conserved. In this regard, all V region sequences contain an internal disulfide loop of around 90 amino acid residues. When the V regions fold into a binding-site, the CDRs are displayed as projecting loop motifs which form an antigen-binding surface. It is generally recognized that there are conserved structural regions of FRs, which influence the folded shape of the CDR loops into certain “canonical” structures-regardless of the precise CDR amino acid sequence. Further, certain FR residues are known to participate in non-covalent interdomain contacts which stabilize the interaction of the antibody heavy and light chains.

A “monoclonal antibody” refers to a homogeneous antibody population wherein the monoclonal antibody is comprised of amino acids (naturally occurring and non-naturally occurring) that are involved in the selective binding of an epitope. Monoclonal antibodies are highly specific, being directed against a single epitope. The term “monoclonal antibody” encompasses not only intact monoclonal antibodies and full-length monoclonal antibodies, but also fragments thereof (such as Fab, Fab′, F(ab′) 2, Fv), single chain (scFv), Nanobodies®, variants thereof, fusion proteins comprising an antigen-binding fragment of a monoclonal antibody, humanized monoclonal antibodies, chimeric monoclonal antibodies, and any other modified configuration of the immunoglobulin molecule that comprises an antigen-binding fragment (epitope recognition site) of the required specificity and the ability to bind to an epitope, including Wnt surrogate molecules disclosed herein. It is not intended to be limited as regards the source of the antibody or the manner in which it is made (e.g., by hybridoma, phage selection, recombinant expression, transgenic animals, etc.). The term includes whole immunoglobulins as well as the fragments etc. described above under the definition of “antibody”.

The proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab′) 2 fragment which comprises both antigen-binding sites. An Fv fragment for use according to certain embodiments of the present disclosure can be produced by preferential proteolytic cleavage of an IgM, and on rare occasions of an IgG or IgA immunoglobulin molecule. Fv fragments are, however, more commonly derived using recombinant techniques known in the art. The Fv fragment includes a non-covalent VH::VL heterodimer including an antigen-binding site which retains much of the antigen recognition and binding capabilities of the native antibody molecule. Inbar et al. (1972) Proc. Nat. Acad. Sci. USA 69:2659-2662; Hochman et al. (1976) Biochem 15:2706-2710; and Ehrlich et al. (1980) Biochem 19:4091-4096.

In certain embodiments, single chain Fv or scFV antibodies are contemplated. For example, Kappa bodies (Ill et al., Prot. Eng. 10:949-57 (1997)); minibodies (Martin et al., EMBO J 13:5305-9 (1994)); diabodies (Holliger et al., PNAS 90:6444-8 (1993)); or Janusins (Traunecker et al., EMBO J 10:3655-59 (1991) and Traunecker et al., Int. J. Cancer Suppl. 7:51-52 (1992)), may be prepared using standard molecular biology techniques following the teachings of the present application with regard to selecting antibodies having the desired specificity. In still other embodiments, bispecific or chimeric antibodies may be made that encompass the ligands of the present disclosure. For example, a chimeric antibody may comprise CDRs and framework regions from different antibodies, while bispecific antibodies may be generated that bind specifically to one or more Fzd receptors through one binding domain and to a second molecule through a second binding domain. These antibodies may be produced through recombinant molecular biological techniques or may be physically conjugated together.

In certain embodiments, the antibodies of the present disclosure may take the form of a Nanobody®. Nanobody® technology was originally developed following the discovery and identification that camelidae (e.g., camels and llamas) possess fully functional antibodies that consist of heavy chains only and therefore lack light chains. These heavy-chain only antibodies contain a single variable domain (VHH) and two constant domains (CH2, CH3). The cloned and isolated single variable domains have full antigen binding capacity and are very stable. These single variable domains, with their unique structural and functional properties, form the basis of “Nanobodies®”. Nanobodies® are encoded by single genes and are efficiently produced in almost all prokaryotic and eukaryotic hosts, e.g., E. coli (see, e.g., U.S. Pat. No. 6,765,087), molds (for example Aspergillus or Trichoderma) and yeast (for example Saccharomyces, Kluyvermyces, Hansenula or Pichia (see, e.g., U.S. Pat. No. 6,838,254). The production process is scalable and multi-kilogram quantities of Nanobodies® have been produced. Nanobodies® may be formulated as a ready-to-use solution having a long shelf life. The Nanoclone® method (see, e.g., WO 06/079372) is a proprietary method for generating Nanobodies® against a desired target, based on automated high-throughput selection of B-cells. Nanobodies® are single-domain antigen-binding fragments of camelid-specific heavy-chain only antibodies. Nanobodies®, also referred to as VHH antibodies, typically have a small size of around 15 kDa.

Another antibody fragment contemplated is a dual-variable domain-immunoglobulin (DVD-Ig) is an engineered protein that combines the function and specificity of two monoclonal antibodies in one molecular entity. A DVD-Ig is designed as an IgG-like molecule, except that each light chain and heavy chain contains two variable domains in tandem through a short peptide linkage, instead of one variable domain in IgG. The fusion orientation of the two variable domains and the choice of linker sequence are critical to functional activity and efficient expression of the molecule. A DVD-Ig can be produced by conventional mammalian expression systems as a single species for manufacturing and purification. A DVD-Ig has the specificity of the parental antibodies, is stable in vivo, and exhibits IgG-like physicochemical and pharmacokinetic properties. DVD-Igs and methods for making them are described in Wu, C., et al., Nature Biotechnology, 25:1290-1297 (2007)).

In certain embodiments, the antibodies or antigen-binding fragments thereof as disclosed herein are humanized. This refers to a chimeric molecule, generally prepared using recombinant techniques, having an antigen-binding site derived from an immunoglobulin from a non-human species and the remaining immunoglobulin structure of the molecule based upon the structure and/or sequence of a human immunoglobulin. The antigen-binding site may comprise either complete variable domains fused onto constant domains or only the CDRs grafted onto appropriate framework regions in the variable domains. Epitope binding sites may be wild type or modified by one or more amino acid substitutions. This eliminates the constant region as an immunogen in human individuals, but the possibility of an immune response to the foreign variable region remains (LoBuglio, A. F. et al., (1989) Proc Natl Acad Sci USA 86:4220-4224; Queen et al., PNAS (1988) 86:10029-10033; Riechmann et al., Nature (1988) 332:323-327). Illustrative methods for humanization of the anti-Fzd or LRP antibodies disclosed herein include the methods described in U.S. Pat. No. 7,462,697.

Another approach focuses not only on providing human-derived constant regions, but modifying the variable regions as well so as to reshape them as closely as possible to human form. It is known that the variable regions of both heavy and light chains contain three complementarity-determining regions (CDRs) which vary in response to the epitopes in question and determine binding capability, flanked by four framework regions (FRs) which are relatively conserved in a given species and which putatively provide a scaffolding for the CDRs. When nonhuman antibodies are prepared with respect to a particular epitope, the variable regions can be “reshaped” or “humanized” by grafting CDRs derived from nonhuman antibody on the FRs present in the human antibody to be modified. Application of this approach to various antibodies has been reported by Sato, K., et al., (1993) Cancer Res 53:851-856; Riechmann, L., et al., (1988) Nature 332:323-327; Verhoeyen, M., et al., (1988) Science 239:1534-1536; Kettleborough, C. A., et al., (1991) Protein Engineering 4:773-3783; Maeda, H., et al., (1991) Human Antibodies Hybridoma 2:124-134; Gorman, S. D., et al., (1991) Proc Natl Acad Sci USA 88:4181-4185; Tempest, P. R., et al., (1991) Bio/Technology 9:266-271; Co, M. S., et al., (1991) Proc Natl Acad Sci USA 88:2869-2873; Carter, P., et al., (1992) Proc Natl Acad Sci USA 89:4285-4289; and Co, M. S. et al., (1992) J Immunol 148:1149-1154. In some embodiments, humanized antibodies preserve all CDR sequences (for example, a humanized mouse antibody which contains all six CDRs from the mouse antibodies). In other embodiments, humanized antibodies have one or more CDRs (one, two, three, four, five, six) which are altered with respect to the original antibody, which are also termed one or more CDRs “derived from” one or more CDRs from the original antibody.

In certain embodiments, the antibodies of the present disclosure may be chimeric antibodies. In this regard, a chimeric antibody is comprised of an antigen-binding fragment of an antibody operably linked or otherwise fused to a heterologous Fc portion of a different antibody. In certain embodiments, the heterologous Fc domain is of human origin. In other embodiments, the heterologous Fc domain may be from a different Ig class from the parent antibody, including IgA (including subclasses IgA1 and IgA2), IgD, IgE, IgG (including subclasses IgG1, IgG2, IgG3, and IgG4), and IgM. In further embodiments, the heterologous Fc domain may be comprised of CH2 and CH3 domains from one or more of the different Ig classes. As noted above with regard to humanized antibodies, the antigen-binding fragment of a chimeric antibody may comprise only one or more of the CDRs of the antibodies described herein (e.g., 1, 2, 3, 4, 5, or 6 CDRs of the antibodies described herein), or may comprise an entire variable domain (VL, VH or both).

The structures and locations of immunoglobulin CDRs and variable domains may be determined by reference to Kabat, E. A. et al., Sequences of Proteins of Immunological Interest. 4th Edition. US Department of Health and Human Services. 1987, and updates thereof, now available on the Internet (immuno.bme.nwu.edu).

In certain embodiments, the antagonist or agonist binding agent binds with a dissociation constant (K_{D}) of about 1 μM or less, about 100 nM or less, about 40 nM or less, about 20 nM or less, or about 10 nM or less. For example, in certain embodiments, a FZD binding agent or antibody described herein that binds to more than one FZD, binds to those FZDs with a K_{D} of about 100 nM or less, about 20 nM or less, or about 10 nM or less. In certain embodiments, the binding agent binds to one or more its target antigen with an EC50 of about 1 μM or less, about 100 nM or less, about 40 nM or less, about 20 nM or less, about 10 nM or less, or about 1 nM 20 or less. KD is inversely proportional to affinity, so the lower the KD value (the lower the concentration), the higher the affinity of the antibody. High-affinity interactions are characterized by low KD, rapid recognition (high Kₒₙ), and strong stability of the formed complex (low K_{off}). Therefore, KD can be used to evaluate antibody affinity or sensitivity. For example, when the KD value is 10⁻⁴ to 10⁻⁶, the antibody sensitivity is micromolar; when the KD value is 10⁻⁷ to 10⁻⁹, the antibody sensitivity is nanomolar; When the KD value is 10⁻¹⁰ to 10⁻¹², its antibody sensitivity is picomolar concentration, when KD value is 10⁻¹³ to 10⁻¹⁵, its antibody sensitivity is femtomolar concentration. In particular embodiments, the K_{D} is less than or about 10⁻⁴, 10⁻⁵, 10⁻⁶, 10⁻⁷, 10⁻⁸, 10⁻⁹, 10⁻¹⁰, 10⁻¹¹, 10⁻¹², 10⁻¹³, 10⁻¹⁴, or 10⁻¹⁵.

The antibodies or other agents of the present invention can be assayed for specific binding by any method known in the art. The immunoassays which can be used include, but are not limited to, competitive and non-competitive assay systems using techniques such as BIAcore analysis, FACS analysis, immunofluorescence, immunocytochemistry, Western blots, radioimmunoassays, ELISA, “sandwich” immunoassays, immunoprecipitation assays, precipitation reactions, gel diffusion precipitin reactions, immunodiffusion assays, agglutination assays, complement-fixation assays, immunoradiometric assays, fluorescent immunoassays, and protein A immunoassays. Such assays are routine and well known in the art (see, e.g., Ausubel et al, eds, 1994, Current Protocols in Molecular Biology, Vol. 1, John Wiley & Sons, Inc., New York, which is incorporated by reference herein in its entirety).

For example, the specific binding of an antibody to a target antigen may be determined using ELISA. An ELISA assay comprises preparing antigen, coating wells of a 96 well microtiter plate with antigen, adding the antibody or other binding agent conjugated to a detectable compound such as an enzymatic substrate (e.g., horse-radish peroxidase or alkaline phosphatase) to the well, incubating for a period of time and detecting the presence of the antigen. In some embodiments, the antibody or agent is not conjugated to a detectable compound, but instead a second conjugated antibody that recognizes the first antibody or agent is added to the well. In some embodiments, instead of coating the well with the antigen, the antibody or agent can be coated to the well and a second antibody conjugated to a detectable compound can be added following the addition of the antigen to the coated well. One of skill in the art would be knowledgeable as to the parameters that can be modified to increase the signal detected as well as other variations of ELISAs known in the art (see e.g., Ausubel et al, eds, 1994, Current Protocols in Molecular Biology, Vol. 1, John Wiley & Sons, Inc., New York at 11.2.1).

The binding affinity of an antibody or other agent to a target antigen and the off-rate of the antibody-antigen interaction can be determined by competitive binding assays. One example of a competitive binding assay is a radioimmunoassay comprising the incubation of labeled antigen (e.g., Fzd, LRP), or fragment or variant thereof, with the antibody of interest in the presence of increasing amounts of unlabeled antigen followed by the detection of the antibody bound to the labeled antigen. The affinity of the antibody and the binding off-rates can be determined from the data by scatchard plot analysis. In some embodiments, BIAcore kinetic analysis is used to determine the binding on and off rates of antibodies or agents. BIAcore kinetic analysis comprises analyzing the binding and dissociation of antibodies from chips with immobilized antigens on their surface.

### III. Wnt Agonists, Wnt Antagonists, and Pharmaceutical Compositions

In some embodiments, the Wnt antagonist or Wnt agonist is an engineered recombinant polypeptide incorporating various epitope binding fragments that bind to various molecules in the Wnt signaling pathway. For example, a Wnt antagonist can be an antibody or fragment thereof that binds to one or more Fzd receptor or an LRP receptor and inhibits Wnt signaling. As another example, a Wnt agonist may comprise an antibody or fragment thereof that binds to one or more Fzd receptor and an antibody or fragment thereof that binds to an LRP receptor (e.g., LRP5 and/or LRP6). The Fzd and LRP antibody fragments (e.g., Fab, scFv, Nanobodies®, etc) may be joined together directly or with various size linkers, on one molecule. Similarly, a polypeptide such as RSPO, may be engineered to contain an antibody or fragment thereof against a tissue specific cell surface antigen, e.g., HTII-280.RSPO may also be administered concurrently or sequentially with an enhancer of the E3 ligases, ZNRF3/RNF43. The E3 ligase enhancer may be an agonist antibody or fragment that binds ZNRF3/RNF43 and enhances the E3 ligase activity.

Conversely, Wnt agonists can also be recombinant polypeptides incorporating epitope binding fragments that bind to various molecules in the Wnt signaling pathway and enhance Wnt signaling. For example, a Wnt agonist can be an antibody or fragment thereof that binds to Fzd receptor and/or an LRP receptor and enhances Wnt signaling. The Fzd and LRP antibody fragments (e.g., Fab, scFv, Nanobodies®, etc) may be joined together directly or with various size linkers, on one molecule.

In certain embodiments, a Wnt agonist or Wnt antagonist binds either or both of LRP5 and/or LRP6. In certain embodiments, a Wnt agonist or Wnt antagonist specifically binds only one Frizzled, i.e., is mono-specific, while in other embodiments, a Wnt agonist or Wnt antagonist binds two or more Frizzleds, i.e., is multi-specific. In particular embodiments, the Wnt agonist or Wnt antagonist is FZD1,2,7-specific, FZD5,8-specific, or FZD4-mono-specific. In particular embodiments, the Wnt agonist is SWAP1, SWAP2, SWAP3, SWAP4, SWAP5, or SWAP6, or a functional variant or fragment thereof. A functional variant or fragment thereof may bind the same targets, e.g., with at least 40%, at least 50%, at least 60-%, at least 70%, at least 80%, at least 90%, at least 100%, or greater binding affinity.

In certain embodiments, a Wnt antagonist antibody or fragment thereof, comprises the CDRs present in an anti-LRP5 or anti-LRP6 antibody disclosed in U.S. patent application Publication No. 20210079089. In certain embodiments, a Wnt antagonist antibody or fragment thereof, comprises a heavy chain and/or light chain present in an anti-LRP5 or anti-LRP6 antibody disclosed in U.S. patent application Publication No. 20210079089. In certain embodiments, a Wnt antagonist antibody or fragment thereof, comprises the CDRs present in an anti-FZD antibody disclosed in U.S. patent application Publication No. 20210087280, PCT patent application publication No. WO2021/003054, U.S. provisional application No. 62/875,073, or U.S. patent application Publication No. 20220275095. In certain embodiments, a Wnt antagonist antibody or fragment thereof, comprises a heavy chain and/or light chain present in an anti-FZD antibody disclosed in U.S. patent application Publication No. 20210087280, PCT patent application publication No. WO2021/003054, U.S. provisional application No. 62/875,073, or U.S. patent application Publication No. 20220275095.

In certain embodiments, a Wnt agonist comprises an antibody or fragment thereof comprising the CDRs present in an anti-LRP5 or anti-LRP6 antibody disclosed in U.S. patent application Publication No. 20210079089. In certain embodiments, a Wnt antagonist comprises an antibody or fragment thereof comprising a variable heavy chain region and/or variable light chain region (or a full heavy chain and/or full light chain) present in an anti-LRP5 or anti-LRP6 antibody disclosed in U.S. patent application Publication No. 20210079089. In certain embodiments, a Wnt agonist comprises an antibody or fragment thereof comprising the CDRs present in an anti-FZD antibody disclosed in U.S. patent application Publication No. 20210087280, PCT patent application publication No. WO2021/003054, or U.S. provisional application No. 62/875,073, or U.S. patent application Publication No. 20220275095. In certain embodiments, a Wnt agonist comprises an antibody or fragment thereof comprising a variable heavy chain region and/or variable light chain region (or a full heavy chain and/or full light chain) present in an anti-FZD antibody disclosed in U.S. patent application Publication No. 20210087280, PCT patent application publication No. WO2021/003054, U.S. provisional application No. 62/875,073, or U.S. patent application Publication No. 20220275095. In certain embodiments, a Wnt agonist comprises an antibody or fragment thereof comprising the CDRs present in an anti-LRP5 or anti-LRP6 antibody disclosed in U.S. patent application Publication No. 20210079089, and an antibody or fragment thereof comprising the CDRs present in an anti-FZD antibody disclosed in U.S. patent application Publication No. 20210087280, PCT patent application publication No. WO2021/003054, U.S. provisional application No. 62/875,073, or U.S. patent application Publication No. 20220275095. In certain embodiments, a Wnt agonist comprises an antibody or fragment thereof comprising a variable heavy chain region and/or variable light chain region (or a full heavy chain and/or full light chain) present in an anti-LRP5 or anti-LRP6 antibody disclosed in U.S. patent application Publication No. 20210079089, and an antibody or fragment thereof comprising a variable heavy chain region and/or variable light chain region (or a full heavy chain and/or full light chain) present in an anti-FZD antibody disclosed in U.S. patent application Publication No. 20210087280, PCT patent application publication No. WO2021/003054, U.S. provisional application No. 62/875,073, or U.S. patent application Publication No. 20220275095. In certain embodiments, a Wnt agonist comprises a Wnt agonist (Wnt surrogate molecule) disclosed in U.S. patent application Publication No. 20200048324, U.S. patent application Publication No. 20200308287, PCT patent application publication No. WO 2020/010308, U.S. provisional patent application No. 62/797,772, or U.S. patent application Publication No. 20210292422 or one or more binding domains thereof. In certain embodiments, a Wnt agonist comprises a variable heavy chain region and/or a variable light chain region (or a full heavy chain and/or full light chain) present in a Wnt agonist (Wnt surrogate molecule) disclosed in U.S. patent application Publication No. 20200048324, U.S. patent application Publication No. 20200308287, PCT patent application publication No. WO 2020/010308, U.S. provisional patent application No. 62/797,772, or U.S. patent application Publication No. 20210292422. In certain embodiments, a Wnt agonist is SWAP1, SWAP2, SWAP3, SWAP4, SWAP 5, or SWAP 6, as disclosed in Table 1, or a variant or fragment thereof.

A mutated RSPO polypeptide, one lacking an active second furin domain (mutated or eliminated) and can no longer bind ZNRF3/RNF43, may be engineered to contain an antibody or fragment thereof against a tissue specific cell surface antigen, e.g., HTII-280. RSPO may also be administered concurrently or sequentially with a further inhibitor of the E3 ligases, ZNRF3/RNF43. The E3 ligase enhancer may be an antagonist antibody or fragment that binds ZNRF3/RNF43 and enhances the E3 ligase activity. The E3 ligase antagonist may also be an siRNA or anti-sense oligomer that inhibits the E3 ligase activity, thus stabilizing the Wnt receptor.

In certain embodiments, a Wnt agonist is a tissue-specific Wnt agonist, e.g., an R-spondin mimetic, e.g., which may inhibit E3 ligase degradation of Fzd polypeptides. In particular embodiments, a Wnt agonist comprises an R-spondin surrogate molecule or binding domain thereof, e.g., as disclosed in U.S. patent application Publication No. 20200024338 or a tissue-specific Wnt signaling enhancing molecule or binding domain thereof, e.g., as disclosed in PCT patent application Publication No. WO2020/014271, U.S. provisional application No. 62/822,731, or U.S. patent application Publication No. US 2021-0380678.

In particular embodiments, a tissue-specific Wnt agonist comprises a tissue targeting molecule or binding region, which specifically binds to a target tissue of interest, e.g., lung tissue. Specific cell types and cells within specific tissue may comprise one or more cell- or tissue-specific surface molecule, such as a cell surface receptor. As used herein, the molecule is said to be cell- or tissue-specific if a greater amount of the molecule is present on the specific cell or tissue type (e.g., lung cells or lung tissue) as compared to one or more other cell or tissue types, or any other cell or tissue type. In certain embodiments, the greater amount is at least two-fold, at least five-fold, at least 10-fold, at least 20-fold, at least 50-fold, or at least 100-fold as compared to the amount in the one or more other cell or tissue types, or any other cell or tissue type. In particular embodiments, the cell-specific surface molecule has increased or enhanced expression on a target organ, tissue or cell type, e.g., an organ, tissue or cell type in which it is desirous to enhance Wnt signaling, e.g., to treat or prevent a disease or disorder, e.g., as compared to one or more other non-targeted organs, tissues or cell types. In certain embodiments, the cell-specific surface molecule is preferentially expressed on the surface of the target organ, tissue or cell type as compared to one or more other organ, tissue or cell types, respectively. For example, in particular embodiments, a cell surface receptor is considered to be a tissue-specific or cell-specific cell surface molecule if it is expressed at levels at least two-fold, at least five-fold, at least 10-fold, at least 20-fold, at least 30-fold, at least 40-fold, at least 50-fold, at least 100-fold, at least 500-fold, or at least 1000-fold higher in the target organ, tissue or cell than it is expressed in one or more, five or more, all other organs, tissues or cells, or an average of all other organs, tissue or cells, respectively. In certain embodiments, the tissue-specific or cell-specific cell surface molecule is a cell surface receptor, e.g., a polypeptide receptor comprising a region located within the cell surface membrane and an extracellular region to which the targeting module can bind. In various embodiments, the methods described herein may be practiced by specifically targeting cell surface molecules that are only expressed on the target tissue or a subset of tissues including the target tissue, or by specifically targeting cell surface molecules that have higher levels of expression on the target tissue as compared to all, most, or a substantial number of other tissues, e.g., higher expression on the target tissue than on at least two, at least five, at least ten, or at least twenty other tissues.

In particular embodiments, the targeting module binds to a tissue-specific surface molecule expressed on a target cell or tissue type of interest, i.e., a cell or tissue type wherein it is desired to enhance or increase Wnt signaling activity. The targeting modules that bind to each tissue-specific surface molecules can be, but are not limited to, antibodies or antigen-binding fragments thereof, peptides, natural ligands of tissue- or cell-specific receptors, or their derivatives, and synthetic small molecules, etc.

In certain embodiments, the tissue targeting molecule is an antibody that binds to a cell surface receptor or marker on the surface of a tissue of interest. In certain embodiments, the tissue is lung.

Pharmaceutical compositions comprising a Wnt antagonist or agonist molecule described herein and one or more pharmaceutically acceptable diluent, carrier, or excipient are also disclosed.

In further embodiments, pharmaceutical compositions comprising a polynucleotide comprising a nucleic acid sequence encoding a Wnt antagonist/agonist molecule described herein and one or more pharmaceutically acceptable diluent, carrier, or excipient are also disclosed. In certain embodiments, the polynucleotides are DNA or mRNA, e.g., a modified mRNA. In particular embodiments, the polynucleotides are modified mRNAs further comprising a 5′ cap sequence and/or a 3′ tailing sequence, e.g., a poly A tail. In other embodiments, the polynucleotides are expression cassettes comprising a promoter operatively linked to the coding sequences.

In further embodiments, pharmaceutical compositions comprising an expression vector, e.g., a viral vector, comprising a polynucleotide comprising a nucleic acid sequence encoding a Wnt antagonist/agonist molecule described herein and one or more pharmaceutically acceptable diluent, carrier, or excipient are also disclosed. In certain embodiments, the nucleic acid sequence encoding the Wnt antagonist molecule and the nucleic acid sequence encoding the Wnt agonist are in the same polynucleotide, e.g., expression cassette.

The present disclosure further contemplates a pharmaceutical composition comprising a cell comprising an expression vector comprising a polynucleotide comprising a promoter operatively linked to a nucleic acid encoding a Wnt antagonist/agonist molecule and one or more pharmaceutically acceptable diluent, carrier, or excipient. In particular embodiments, the pharmaceutical composition further comprises a cell comprising an expression vector comprising a polynucleotide comprising a promoter operatively linked to a nucleic acid sequence encoding a Wnt antagonist and a Wnt agonist. In certain embodiments, the nucleic acid sequence encoding the Wnt antagonist molecule and the nucleic acid sequence encoding the Wnt agonist molecule are present in the same polynucleotide, e.g., expression cassette and/or in the same cell. In particular embodiments, the cell is a heterologous cell or an autologous cell obtained from the subject to be treated.

In particular embodiments, the cell is a stem cell, e.g., an adipose-derived stem cell or a hematopoietic stem cell. The present disclosure contemplates pharmaceutical compositions comprising a first molecule for delivery of a Wnt antagonist molecule as a first active agent, and a Wnt agonist as a second agent. The first and second molecule may be the same type of molecule or different types of molecules. For example, in certain embodiments, the first and second molecule may each be independently selected from the following types of molecules: polypeptides, small organic molecules, nucleic acids encoding the first or second active agent (optionally DNA or mRNA, optionally modified RNA), vectors comprising a nucleic acid sequence encoding the first or second active agent (optionally expression vectors or viral vectors), and cells comprising a nucleic acid sequence encoding the first or second active agent (optionally an expression cassette).

The subject molecules, alone or in combination, can be combined with pharmaceutically-acceptable carriers, diluents, excipients and reagents useful in preparing a formulation that is generally safe, non-toxic, and desirable, and includes excipients that are acceptable for mammalian, e.g., human or primate, use. Such excipients can be solid, liquid, semisolid, or, in the case of an aerosol composition, gaseous. Examples of such carriers, diluents and excipients include, but are not limited to, water, saline, Ringer's solutions, dextrose solution, and 5% human serum albumin. Supplementary active compounds can also be incorporated into the formulations. Solutions or suspensions used for the formulations can include a sterile diluent such as water for injection, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates; detergents such as Tween 20 to prevent aggregation; and compounds for the adjustment of tonicity such as sodium chloride or dextrose. The pH can be adjusted with acids or bases, such as hydrochloric acid or sodium hydroxide. In particular embodiments, the pharmaceutical compositions are sterile.

Pharmaceutical compositions may further include sterile aqueous solutions or dispersions and sterile powders for the extemporaneous preparation of sterile injectable solutions or dispersion. For intravenous administration, suitable carriers include physiological saline, bacteriostatic water, or phosphate buffered saline (PBS). In some cases, the composition is sterile and should be fluid such that it can be drawn into a syringe or delivered to a subject from a syringe. In certain embodiments, it is stable under the conditions of manufacture and storage and is preserved against the contaminating action of microorganisms such as bacteria and fungi. The carrier can be, e.g., a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the internal compositions can be brought about by including in the composition an agent which delays absorption, for example, aluminum monostearate and gelatin.

Sterile solutions can be prepared by incorporating the Wnt antagonist/agonist antibody or antigen-binding fragment thereof (or encoding polynucleotide or cell comprising the same) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

In one embodiment, the pharmaceutical compositions are prepared with carriers that will protect the antibody or antigen-binding fragment thereof against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially. Liposomal suspensions can also be used as pharmaceutically acceptable carriers. These can be prepared according to methods known to those skilled in the art.

It may be advantageous to formulate the pharmaceutical compositions in dosage unit form for ease of administration and uniformity of dosage. Dosage unit form as used herein refers to physically discrete units suited as unitary dosages for the subject to be treated; each unit containing a predetermined quantity of active antibody or antigen-binding fragment thereof calculated to produce the desired therapeutic effect in association with the required pharmaceutical carrier. The specification for the dosage unit forms are dictated by and directly dependent on the unique characteristics of the antibody or antigen-binding fragment thereof and the particular therapeutic effect to be achieved, and the limitations inherent in the art of compounding such an active antibody or antigen-binding fragment thereof for the treatment of individuals.

The pharmaceutical compositions can be included in a container, pack, or dispenser, e.g., a syringe, e.g., a prefilled syringe, together with instructions for administration.

The pharmaceutical compositions of the present disclosure encompass any pharmaceutically acceptable salts, esters, or salts of such esters, or any other compound which, upon administration to an animal comprising a human, is capable of providing (directly or indirectly) the biologically active antibody or antigen-binding fragment thereof.

The present disclosure includes pharmaceutically acceptable salts of a Wnt antagonist/agonist molecule described herein. The term “pharmaceutically acceptable salt” refers to physiologically and pharmaceutically acceptable salts of the compounds of the present disclosure: i.e., salts that retain the desired biological activity of the parent compound and do not impart undesired toxicological effects thereto. A variety of pharmaceutically acceptable salts are known in the art and described, e.g., in “Remington's Pharmaceutical Sciences”, 17th edition, Alfonso R. Gennaro (Ed.), Mark Publishing Company, Easton, PA, USA, 1985 (and more recent editions thereof), in the “Encyclopedia of Pharmaceutical Technology”, 3rd edition, James Swarbrick (Ed.), Informa Healthcare USA (Inc.), NY, USA, 2007, and in J. Pharm. Sci. 66:2 (1977). Also, for a review on suitable salts, see “Handbook of Pharmaceutical Salts: Properties, Selection, and Use” by Stahl and Wermuth (Wiley-VCH, 2002). Pharmaceutically acceptable base addition salts are formed with metals or amines, such as alkali and alkaline earth metals or organic amines.

Metals used as cations comprise sodium, potassium, magnesium, calcium, and the like. Amines comprise N—N′-dibenzylethylenediamine, chloroprocaine, choline, diethanolamine, dicyclohexylamine, ethylenediamine, N-methylglucamine, and procaine (see, for example, Berge et al., “Pharmaceutical Salts,” J. Pharma Sci., 1977, 66, 119). The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present disclosure.

In some embodiments, the pharmaceutical composition provided herein comprise a therapeutically effective amount of a Wnt antagonist/agonist molecule or pharmaceutically acceptable salt thereof in admixture with a pharmaceutically acceptable carrier, diluent and/or excipient, for example saline, phosphate buffered saline, phosphate and amino acids, polymers, polyols, sugar, buffers, preservatives and other proteins. Exemplary amino acids, polymers and sugars and the like are octylphenoxy polyethoxy ethanol compounds, polyethylene glycol monostearate compounds, polyoxyethylene sorbitan fatty acid esters, sucrose, fructose, dextrose, maltose, glucose, mannitol, dextran, sorbitol, inositol, galactitol, xylitol, lactose, trehalose, bovine or human serum albumin, citrate, acetate, Ringer's and Hank's solutions, cysteine, arginine, carnitine, alanine, glycine, lysine, valine, leucine, polyvinylpyrrolidone, polyethylene and glycol. Preferably, this formulation is stable for at least six months at 4° C.

In some embodiments, the pharmaceutical composition provided herein comprises a buffer, such as phosphate buffered saline (PBS) or sodium phosphate/sodium sulfate, tris buffer, glycine buffer, sterile water and other buffers known to the ordinarily skilled artisan such as those described by Good et al. (1966) Biochemistry 5:467. The pH of the buffer may be in the range of 6.5 to 7.75, preferably 7 to 7.5, and most preferably 7.2 to 7.4.

### IV. Methods of Use

The present disclosure also provides methods for using the Wnt antagonist/agonist molecules, e.g., to modulate a Wnt signaling pathway, e.g., to increase or decrease Wnt signaling, and the administration of a Wnt antagonist/agonist molecule in a variety of therapeutic settings. Provided herein are methods of treatment using a Wnt antagonist/agonist molecule. In one embodiment, a Wnt antagonist/agonist molecule is provided to a subject having a disease involving inappropriate or deregulated Wnt signaling.

In certain embodiments, a Wnt antagonist/agonist molecule may be used to block or enhance a Wnt signaling pathway in a tissue or a cell. Antagonizing the Wnt signaling pathway may include decreasing or inhibiting Wnt signaling in a cell or tissue. Agonizing the Wnt signaling pathway may include, for example, increasing Wnt signaling or enhancing Wnt signaling in a tissue or cell. Thus, in some aspects, the present disclosure provides a method for antagonizing/agonizing a Wnt signaling pathway in a cell, comprising contacting the tissue or cell with an effective amount of a Wnt antagonist/agonist molecule or pharmaceutically acceptable salt thereof disclosed herein, wherein the Wnt antagonist/agonist molecule is a Wnt signaling pathway antagonist/agonist. In some embodiments, contacting occurs in vitro, ex vivo, or in vivo. In particular embodiments, the cell is a cultured cell or a cell obtained from a patient, and the contacting occurs in vitro or ex vivo. In particular, culturing both iPSC-derived and primary lung epithelial cells, including AT2 cells, and modulating Wnt signaling in these cells via agonism and/or antagonism in preparation of transplanting them into diseased lungs or into patients with lung disease. In certain embodiments, the cells are obtained from a patient, contacted with a Wnt antagonist and/or Wnt agonist molecule ex vivo, and then transplanted into the patient's lung(s). In certain embodiments, the cells obtained from the patient are non-diseased. In certain embodiments, cells (e.g., lungs cells, such as, e.g., primary lung epithelial cells) obtained from a donor are contacted with a Wnt antagonist and/or Wnt agonist molecule ex vivo, and then transplanted into a patient. In certain embodiments, a lung obtained from a donor is contacted with a Wnt antagonist and/or Wnt agonist molecule ex vivo, and then transplanted into a patient.

The Wnt antagonist/agonist molecule may be used for the treatment of pulmonary disorders. In particular, active Wnt signaling can promote and is necessary for mesenchymal cell mediated formation of fibrosis, impacting mesenchymal cell proliferation, apoptosis, and alteration of the extracellular matrix production. Additionally, agonizing Wnt signaling promotes the proliferation of the pulmonary specific progenitor AT2/AEP (alveolar epithelial progenitor) in addition to myofibroblasts. Hyperproliferation of AT2 cells, can contribute to the reduction in ATI cells. Therefore, antagonizing Wnt signaling not only inhibits the formation of fibrosis in lung mesenchymal tissue but also allows the differentiation of AT2 cells into AT1 cells, and repair of lung tissue.

In certain embodiments, a Wnt agonist may be used to promote or increase proliferation of AT2 cells.

In certain embodiments, a Wnt antagonist may be used to promote or increase differentiation of AT2 cells into AT1 cells.

In certain embodiments, epithelial cells, e.g., AT2 epithelial cells may be first contacted with a Wnt agonist to promote or increase proliferation of AT2 cells, and then contacted with a Wnt antagonist to promote or increase differentiation of AT2 cells to AT cells.

In certain embodiments, the Wnt agonist/antagonist molecule(s) may be used to impact AT2/AT1 transition state cells and/or aberrant basaloid cells present in pulmonary fibrosis diseases and COPD. This could be to promote their proper self-renewal and/or differentiation into AT1 cells.

In certain embodiments, the Wnt agonist/antagonist molecule(s) may be designed to impact immune cells including macrophages to promote an anti-inflammatory phenotype that reduces tissue inflammation and promotes tissue repair.

The Wnt antagonist and agonist may be administered concurrently or sequentially. The antagonist and agonist may be separate molecules or may be constructed on a single molecule. With concurrent treatment of both antagonist and agonist, it may be possible to link the antagonist and agonist activities together in one molecule by using protease cleavable linkers e.g., uPA. Another version of linking these opposing activities in one molecule would be to use a cell surface antigen that is expressed in all cell types of the lung coupled to a specific FZD receptor(s) binder and an LRP5/6 binder. Antagonism would occur in the cell that does not express the specific FZD because the binder would bind the cell surface antigen and LRP, but in the cell that expressed the cell surface binder and the specific FZD receptor(s) and LRP5/6, an active signaling complex could occur, resulting in antagonism and agonism embodied in one molecule that is specific to the lung.

In a further embodiment, the antagonist and/or agonist molecule may also incorporate a tissue-targeting moiety, e.g., an antibody or fragment thereof that recognizes a pulmonary tissue specific receptor or cell surface molecule.

In certain embodiments, the antagonist and agonist are either sequentially or concurrently administered. In a first dosing schedule, a general or specific antagonist is administered first and then followed by application of a cell type specific agonist. It might be appropriate to apply the agonist in pulsatile doses so that the AT2 cells proliferate, but upon a reduction in signaling, some fraction will then spontaneously differentiate into ATI cells. Then, the second and any subsequent pulses would be used to repeat this process. In the second dosing schedule case, either a general or cell type specific-antagonist would be administered at the same time as a cell type-specific agonist. In this scheme, the antagonist could limit all Wnt signaling either at the ligand or receptor level, but this could be overcome in a specific cell type by using a cell type specific agonist that functions at the receptor level. A third scheme could employ sequential or concurrent combination treatment followed by repeated application of an antagonist. In a fourth scheme, an agonist is administered first followed by application of an antagonist, with the potential for both to be cell type specific. In other embodiments, the antagonist alone is administered to treat tissue damage in IPF, while the agonist is administered alone for treatment of damage as the result or COPD.

The present invention also provides for combination treatment with known treatments for IPF and/or COPD. For example, the Wnt antagonist/agonist can be combined with several known therapies for pulmonary fibrosis, including, but not limited to, oxygen therapy, pulmonary rehabilitation, nintedanib (Ofev®), pirfenidone (Esbriet®, Pirfenex®, Pirespa®), corticosteroids (prednisone), mycopholate motetil/mycopholic acid (CellCept®), azathioprine (Imuran®), methotrexate, cyclophosphamide, cyclosporine, rapamycin (sirolimus), tacrolimus, tankyrase inhibitors (e.g., XAV939) and porcupine (PORCN) inhibitors (e.g., WNT974, LKG974, IWP-2, C59, etc.). For COPD, the Wnt antagonist/agonist can be combined with therapies including, but not limited to, short acting bronchodilators such as albuterol (Pro Air® HFA, Ventolin® HFA), levalbuterol (Xopenex® HFA), and ipratropium (Atrovent®); long acting bronchodilators such as tiotropium (Spiriva®), salmeterol (Serevent®), formoterol (Foradil®, Perforomist®), arformoterol (Brovana®), indacaterol (Arcapta®), and aclidinium (Tudorza®); inhaled steroids such as fluticasone (Flovent® HFA, Flonase®) and budesonide (Plumicort Flexhaler®, Uceris®); combination inhalers such as fluticasone (Advair®) and formoterol and budesonide (Symbicort®); oral steroids; phosphodiesterase-4 inhibitors such as roflumilast (Daliresp®); theophylline; antibiotics; oxygen therapy; and pulmonary rehabilitation. The above therapeutic drugs can be administered sequentially or concurrently with the molecules of the present invention.

The methods of the present invention can be used to treat a variety of pulmonary disorders, including, but not limited to, idiopathic pulmonary fibrosis, cryptogenic organizing pneumonia, desquamative interstitial pneumonitis, nonspecific interstitial pneumonitis, hypersensitivity pneumonitis, acute interstitial pneumonitis, interstitial pneumonia, systemic sclerosis-associated pulmonary fibrosis, sarcoidosis, asbestosis-induced fibrosis, lung injury as the result of acute and chronic lung infections (e.g., viral, bacterial, fungal), pneumonia, aspiration injuries, sepsis, acute respiratory distress syndrome, and neonatal lung development. Additionally, it is contemplated that the methods of the present invention can also be used to treat chronic obstructive pulmonary disease (COPD), including but not limited to, emphysema, chronic asthma, and chronic bronchitis.

The therapeutic agent (e.g., a Wnt antagonist/agonist) may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease. In some embodiments, the subject method results in a therapeutic benefit, e.g., preventing the development of a disorder, halting the progression of a disorder, reversing the progression of a disorder, etc. In some embodiments, the subject method comprises the step of detecting that a therapeutic benefit has been achieved. The ordinarily skilled artisan will appreciate that such measures of therapeutic efficacy will be applicable to the particular disease being modified, and will recognize the appropriate detection methods to use to measure therapeutic efficacy.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the present disclosure have been described herein for purposes of illustration, various modifications may be made without deviating from the spirit and scope of the present disclosure. Accordingly, the present disclosure is not limited except as by the appended claims.

The broad scope of this invention is best understood with reference to the following examples, which are not intended to limit the inventions to the specific embodiments.

### EXAMPLES

### I. General Methods

Standard methods in molecular biology are described. Maniatis et al. (1982) Molecular Cloning, A Laboratory Manual, Cold Spring Harbor Laboratory Press, Cold Spring Harbor, N.Y.; Sambrook and Russell (2001) Molecular Cloning, 3rd ed., Cold Spring Harbor Laboratory Press, Cold Spring Harbor, N.Y.; Wu (1993) Recombinant DNA, Vol. 217, Academic Press, San Diego, Calif. Standard methods also appear in Ausbel et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described. Coligan et al. (2000) Current Protocols in Protein Science, Vol. 1, John Wiley and Sons, Inc., New York. Chemical analysis, chemical modification, post-translational modification, production of fusion proteins, glycosylation of proteins are described. See, e.g., Coligan et al. (2000) Current Protocols in Protein Science, Vol. 2, John Wiley and Sons, Inc., New York; Ausubel et al. (2001) Current Protocols in Molecular Biology, Vol. 3, John Wiley and Sons, Inc., NY, N.Y., pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391. Production, purification, and fragmentation of polyclonal and monoclonal antibodies are described. Coligan et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra. Standard techniques for characterizing ligand/receptor interactions are available. See, e.g., Coligan et al. (2001) Current Protocols in Immunology, Vol. 4, John Wiley, Inc., New York.

Methods for flow cytometry, including fluorescence activated cell sorting detection systems (FACS®), are available. See, e.g., Owens et al. (1994) Flow Cytometry Principles for Clinical Laboratory Practice, John Wiley and Sons, Hoboken, N.J.; Givan (2001) Flow Cytometry, 2^{nd} ed.; Wiley-Liss, Hoboken, N.J.; Shapiro (2003) Practical Flow Cytometry, John Wiley and Sons, Hoboken, N.J. Fluorescent reagents suitable for modifying nucleic acids, including nucleic acid primers and probes, polypeptides, and antibodies, for use, e.g., as diagnostic reagents, are available. Molecular Probes (2003) Catalogue, Molecular Probes, Inc., Eugene, Oreg.; Sigma-Aldrich (2003) Catalogue, St. Louis, Mo.

Standard methods of histology of the immune system are described. See, e.g., Muller-Harmelink (ed.) (1986) Human Thymus: Histopathology and Pathology, Springer Verlag, New York, N.Y.; Hiatt, et al. (2000) Color Atlas of Histology, Lippincott, Williams, and Wilkins, Phila, Pa.; Louis, et al. (2002) Basic Histology: Text and Atlas, McGraw-Hill, New York, N. Y.

Software packages and databases for determining, e.g., antigenic fragments, leader sequences, protein folding, functional domains, glycosylation sites, and sequence alignments, are available. See, e.g., GenBank, Vector NTI® Suite (Informax, Inc, Bethesda, Md.); GCG Wisconsin Package (Accelrys, Inc., San Diego, Calif.); DeCypher® (TimeLogic Corp., Crystal Bay, Nev.); Menne et al. (2000) Bioinformatics 16:741-742; Menne et al. (2000) Bioinformatics Applications Note 16:741-742; Wren et al. (2002) Comput. Methods Programs Biomed. 68:177-181; von Heijne (1983) Eur. J. Biochem. 133:17-21; von Heijne (1986) Nucleic Acids Res. 14:4683-4690.

### II. Single Cell RNA-Seq Analysis

To identify additional cell type specific cell surface expressed gene products, and to specifically profile alveolar epithelial cells and mesenchymal cell populations that can be used for cell type specific targeting, tissue is collected from lungs of normal, IPF, and COPD patients and dissociated to perform single-cell RNA sequencing (scRNA-seq). General procedures are described in the art, e.g., Wang, Y. et al. (2018) PNAS 115:2407-2412; Zepp, J. et al. (2017) Cell 170:1134-1148; Adams et al., (2020) supra; Haberman et al., (2020) supra. Genes specific to the individual cell subpopulations are cross-referenced with known cell surface gene products. The genes that demonstrate expression in specific lung cell subpopulation are assessed for expression in other tissues using public data repositories such as NCBI.

### III. In Vitro Assay to Screen Wnt Antagonists and Agonists

To screen for a Wnt antagonist, primary human lung fibroblasts from healthy donor and IPF patients are obtained and stimulated with TGF-β1 to measure cell proliferation, migration (scratch assay), extra-cellular matrix production (Colla1) and myofibroblast gene expression (αSMA/ACTA2). Additionally, the Wnt antagonist is used to determine whether it can reduce TGFβ1-induced fibrogensis. (see, e.g., Conte, E. et al. (2014) Eur J Pharm Sci. 58:13-19).

Primary human lung AT2 alveolar epithelial cells are derived from healthy donor and IPF patients. Antagonist applied to the AT2 cells promotes and enhances the differentiation into AT1 cells.

For the screening of Wnt agonists, primary human AT2 cells (from healthy donor, IPF patients or COPD patients), primary mouse AT2 cells, the mouse AT2 cell line (MLE12), or the human alveolar epithelial carcinoma cell line, A549 are used. The cells are treated with Wnt agonists comprising a Fzd receptor antibody or fragment thereof combined with an LRP antibody or fragment thereof. Alternatively, Wnt agonists can also be constructed with an antibody or fragment thereof binding to a tissue specific antigen combined with a RSPO mutant polypeptide that reduces E3 ligase activity. Analysis of agonist activities include cell proliferation, migration (scratch assay), Wnt/β-catenin target gene expression (Axin-2, Cyclin D1) and STF activity.

### IV. Precision-Cut Lung Slice (PCLS) Culture

For an ex vivo study, lung slice culture from healthy donor, pulmonary fibrosis and COPD patients is used. The Wnt antagonist and agonist is added alone or sequentially or concurrently to determine the effects on the fibroblast/myofibroblast proliferation, matrix production, alveolar epithelial cell proliferation (AT1 and AT2 cells) and AT1/AT2 cell fate trans-differentiation, and impacts on resident or infiltrating immune cells (see, e.g., Alsafadi, H. et al. (2017) Am J Physiol Lung Cell Mol Physiol. 12:896-902).

### V. Ex vivo Alveolar Organoid Culture

For ex vivo studies, 3D organoid cultures (see, e.g., Zacharias, W. et al. (2018) Nature 555:251-257 and Barkauskas et al. (2017) Development 144:986-997) were constructed by combining primary human lung fibroblasts from healthy, pulmonary fibrosis, or COPD patients with alveolar epithelial cells (AT2 and AEP cells) from patients. Organoids were cultured in SAGM medium with growth factor free Matrigel. The Wnt antagonist and agonist were added in a sequential or concurrent manner to determine the effect on the fibroblast/myofibroblast proliferation, matrix production, alveolar epithelial cell proliferation and AT1/AT2 cell fate trans-differentiation. Alveolar organoids can also be cultured directly from human distal lung cells including purified AT2 cells (Kobayashi et al., (2020) supra).

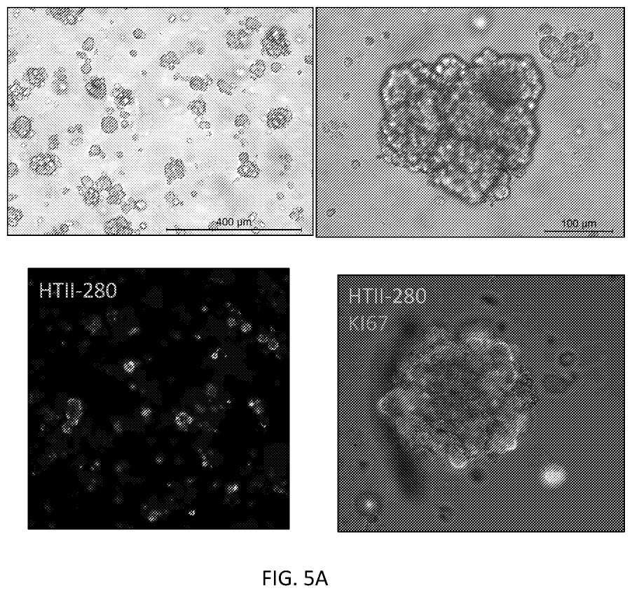

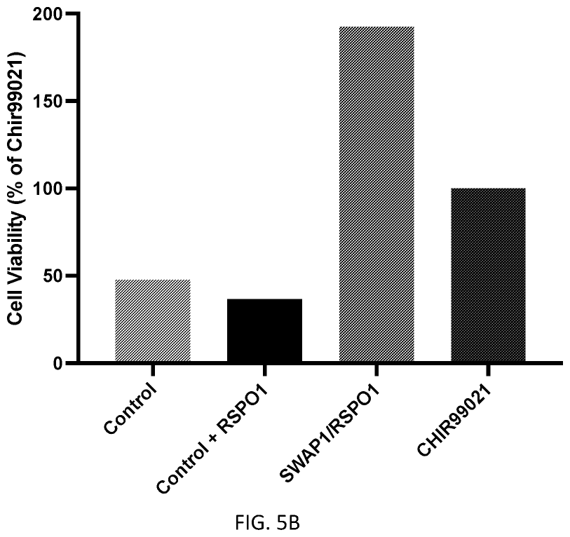

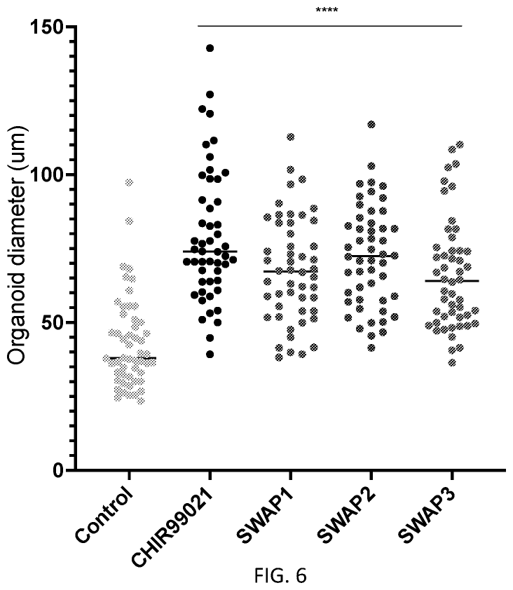

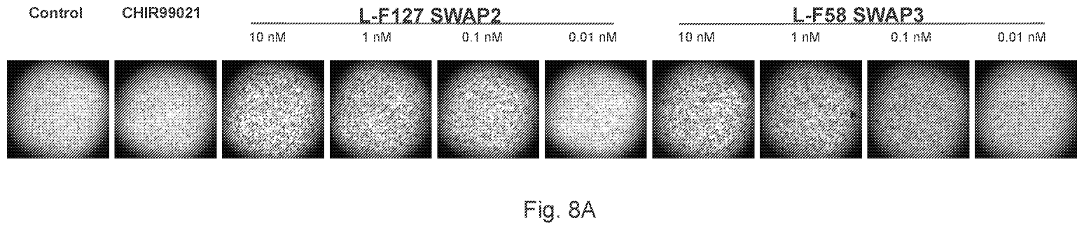

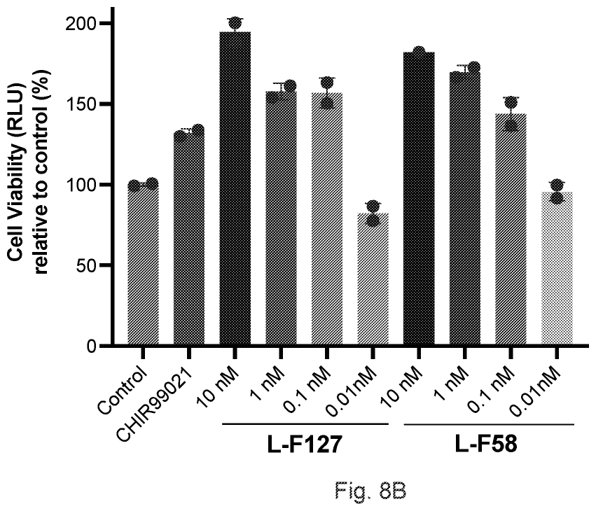

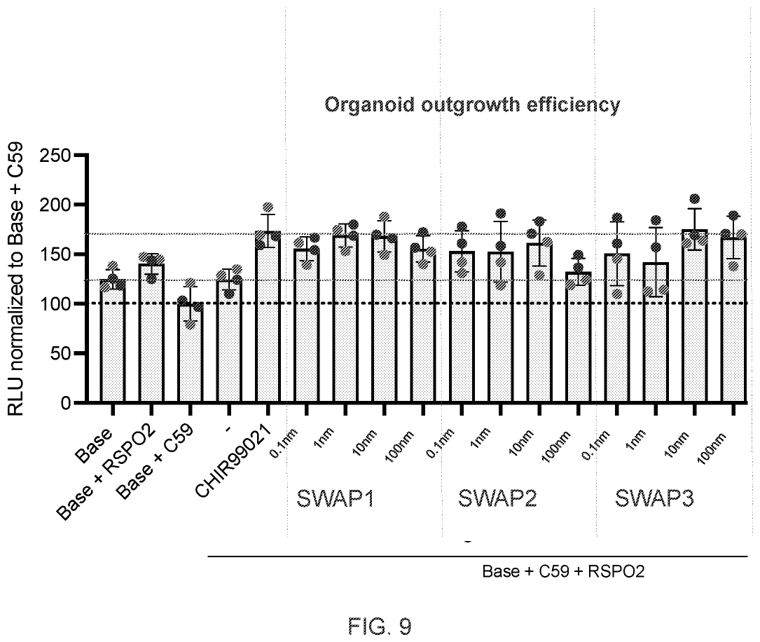

The ability of various WNT agonist SWAPs to promote AT2 self-renewal and alveolar organoid growth was demonstrated (FIGS. 5A and 5B, FIG. 6, FIG. 8, and FIG. 9). In all of the AT2 cell organoid experiments, the Wnt signaling inhibitor, C59, was used to prevent endogenous secretion of Wnt ligands. Treatment with RSPO1 or RSPO2 alone did not enhance human AT2 culture growth, while treatment with the combination of a multi-FZD-specific SWAP, SWAP1, with RSPO1 led to an enhancement of human AT2 cell organoid growth that surpassed the growth observed upon treatment with the small molecule Wnt signaling activator, Chir99021 (FIGS. 5A and 5B). AT2 cell organoid growth was assessed with the CellTiter Glo assay. Furthermore, in the absence of RSPO, subfamily and mono-specific SWAP-mediated Wnt signaling agonist with SWAP2-4 led to enhanced human AT2 cell organoid growth, as indicated by an increase in organoid size (FIG. 6), and SWAP2 and SWAP3 induced human AT2 organoid culture outgrowth/viability as assessed by the CellTiter Glo assay in a concentration-dependent manner, and this was also reflected in the increased numbers of organoids and organoid size by microscopy (FIG. 8.). At the 10 nM concentration, SWAP2 and SWAP3 were more effective than the small molecule Wnt pathway activator, Chir99021 (FIG. 8). Furthermore, SWAP1, SWAP2, and SWAP3 treatment in combination with RSPO2 led to the expansion of mouse AT2 organoid cultures (FIG. 9). Together, these results highlight that agonism through FZD1,2,7,5,8 (SWAP1), FZD1,2,7 (SWAP2), and FZD5,8 (SWAP3), and through FZD4 alone (SWAP4) promotes the self-renewal and expansion of human AT2 cells. In particular, the ability of SWAP3 (specific to FZD5,8 and LRP6) to promote human AT2 cell organoid growth was consistent with the analysis of Wnt receptor expression in published scRNA-seq data from Adams et al., et al. (2020) supra where FZD5 was found to be enriched in the alveolar epithelial cells. The data presented here suggests that a mono-FZD5 SWAP would promote AT2 cell proliferation and self-renewal.

The SWAPS tested included: SWAP1, a FZD-1,2,5,7,8-specific SWAP; SWAP2, a FZD1,2,7-specific SWAP; SWAP3, a FZD5,8-specific SWAP; SWAP4, a FZD4-mono-specific SWAP; and SWAP5, a FZD-1,2,5,7,8-specific SWAP; SWAP6, a FZD1,2,7-specific SWAP. Each of these SWAPS includes an anti-FZD antibody with an LRP6 VHH fused to the N-terminus of the antibody light chains. The sequences of the two heavy chains and two light chains present in each of the SWAPS are shown in Table 1.

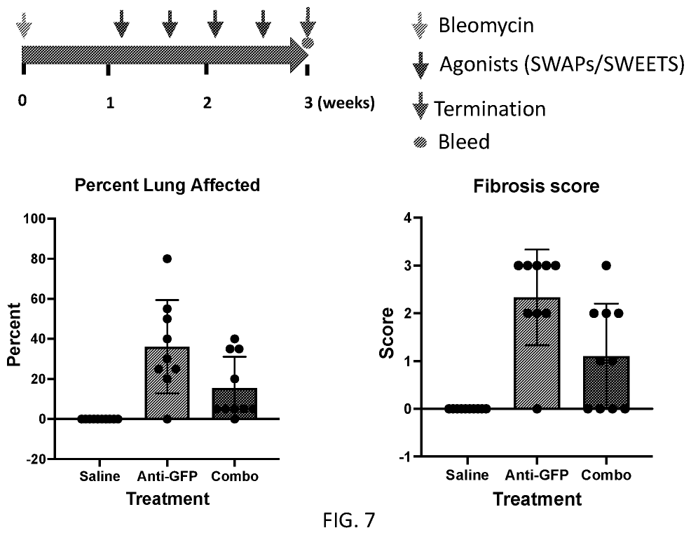

TABLE 1
SWAP sequences

| WNT | SEQ ID | |
|---|---|---|
| AGONIST | NO: | SEQUENCES |
| SWAP5 | 1 | EVQLVQSGAEVKKPGASVKVSCKASGYTFTSYGISWVRQAPG |
| heavy | | QGLEWMGWISAYNGNTNYAQKLQGRVTMTTDTSTSTAYME |
| chain | | LRSLRSDDTAVYYCASSKEKATYYYGMDVWGQGTTVTVSSAS |
| (FIG. 7) | | TKGPSVFPLAPSSKSTSGGTAALGCLVKDYFPEPVTVSWNSGALTSGV |
| | | HTFPAVLQSSGLYSLSSVVTVPSSSLGTQTYICNVNHKPSNTKVDKKVE |
| | | PKSCDKTHTCPPCPAPEAAGGPSVFLFPPKPKDTLMISRTPEVTCVV |
| | | VDVSHEDPEVKFNWYVDGVEVHNAKTKPREEQYNSTYRVVSVLTVLH |
| | | QDWLNGKEYKCKVSNKALGAPIEKTISKAKGQPREPQVYTLPPSRE |
| | | EMTKNQVSLTCLVKGFYPSDIAVEWESNGQPENNYKTTPPVL |
| | | DSDGSFFLYSKLTVDKSRWQQGNVFSCSVMHEALHNHYTQK |
| | | SLSLSPGK |
| SWAP5 | 2 | DVQLVESGGGLVQAGGSLRLACAGSGRIFAIYDIAWYRHPPGNQR |
| light chain | | ELVAMIRPVVTEIDYADSVKGRFTISRNNAMKTVYLQMNNLKPED |
| (FIG. 7) | | TAVYYCNAKRPWGSRDEYWGQGTQVTVSSGSGSGQAVVLQEPSL |
| | | SVSPGGTVTLTCGLSSGSVSTNYYPSWYQQTPGQAPRTLIYYTNT |
| | | RSSDVPERFSGSIVGNKAALTITGAQPDDESVYFCLLYLGRGIWVF |
| | | GGGTKLTVLGQPKAAPSVTLFPPSSEELQANKATLVCLISDFYPG |
| | | AVTVAWKADSSPVKAGVETTTPSKQSNNKYAASSYLSLTPEQWK |
| | | SHRSYSCQVTHEGSTVEKTVAPTECS |
| SWAP1 | 3 | EVQLVESGGGLVQPGGSLRLSCAASGFTFTSYYISWVRQAPG |
| heavy | | KGLEWVAEISPYSGSTYYADSVKGRFTISADTSKNTAYLQMNS |
| chain | | LRAEDTAVYYCALRARPPIRLHPRGSVMDYWGQGTLVTVSSG |
| (FIGs. 5A | | GGGSGGGGSGGGGSEVQLVESGGGLVQPGGSLRLSCAASGFTFSHY |
| and 5B) | | TLSWVRQAPGKGLEWVSVISGDGSYTYYADSVKGRFTISSDNSKNTLYL |
| | | QMNSLRAEDTAVYYCARNFIKYVFANWGQGTLVTVSSASTKGPSVFPL |
| | | APSSKSTSGGTAALGCLVKDYFPEPVTVSWNSGALTSGVHTFPAVLQS |
| | | SGLYSLSSVVTVPSSSLGTQTYICNVNHKPSNTKVDKKVEPKSCDKTH |
| | | TCPPCPAPEAAGGPSVFLFPPKPKDTLMISRTPEVTCVVVDVSHEDP |
| | | EVKFNWYVDGVEVHNAKTKPREEQYNSTYRVVSVLTVLHQDWLNGK |
| | | EYKCKVSNKALGAPIEKTISKAKGQPREPQVYTLPPSREEMTKNQ |
| | | VSLTCLVKGFYPSDIVEWESNGQPENNYKTTPPVLDSDGSFF |
| | | LYSKLTVDKSRWQQGNVFSCSVMHEALHNHYTQKSLSLSPG |
| | | K |
| SWAP1 | 4 | DIQMTQSPSSLSASVGDRVTITCRASQDVSTAVAWYQQKPGKAPK |
| light chain | | LLIYSASFLYSGVPSRFSGSGSGTDFTLTISSLQPEDFATYYCQQSY |
| (FIGs. 5A | | TTPPTFGQGTKVEIKGGGGSGGGGSGGGGSDIELTQPPSVSVAPG |
| and 5B) | | QTARISCSGDNIGSFYVHWYQQKPGQAPVLVIYDKSNRPSGIPER |
| | | FSGSNSGNTATLTISGTQAEDEADYYCQSYANTLSLVFGGGTKLT |
| | | VLGQPKAAPSVTLFPPSSEELQANKATLVCLISDFYPGAVTVAWK |
| | | ADSSPVKAGVETTTPSKQSNNKYAASSYLSLTPEQWKSHRSYSC |
| | | QVTHEGSTVEKTVAPTECS |
| SWAP2 | 5 | EVQLVESGGGLVQPGGSLRLSCAASGFTFTSYYISWVRQAPG |
| heavy | | KGLEWVAEISPYSGSTYYADSVKGRFTISADTSKNTAYLQMNS |
| chain | | LRAEDTAVYYCALRARPPIRLHPRGSVMDYWGQGTLVTVSS |
| (FIG. 6) | | GGGGSGGGGSGGGGSEVQLVESGGGLVQPGGSLRLSCAASGFNISS |
| | | SYIHWVRQAPGKGLEWVAYIYSSYGSTYYADSVKGRFTISADTSKNTAY |
| | | LQMNSLRAEDTAVYYCARASWYALDYWGQGTLVTVSSASTKGPSVFP |
| | | LAPSSKSTSGGTAALGCLVKDYFPEPVTVSWNSGALTSGVHTFPAVLQ |
| | | SSGLYSLSSVVTVPSSSLGTQTYICNVNHKPSNTKVDKKVEPKSCDKT |
| | | HTCPPCPAPEAAGGPSVFLFPPKPKDTLMISRTPEVTCVVVDVSHED |
| | | PEVKFNWYVDGVEVHNAKTKPREEQYNSTYRVVSVLTVLHQDWLNG |
| | | KEYKCKVSNKALGAPIEKTISKAKGQPREPQVYTLPPSREEMTKN |
| | | QVSLTCLVKGFYPSDIAVEWESNGQPENNYKTTPPVLDSDGSF |
| | | FLYSKLTVDKSRWQQGNVFSCSVMHEALHNHYTQKSLSLSP |
| | | GK |
| SWAP2 | 6 | DIQMTQSPSSLSASVGDRVTITCRASQDVSTAVAWYQQKPGKAPK |
| light chain | | LLIYSASFLYSGVPSRFSGSGSGTDFTLTISSLQPEDFATYYCQQSY |
| (FIG. 6) | | TTPPTFGQGTKVEIKGGGGSGGGGSGGGGSDIQMTQSPSSLSASV |
| | | GDRVTITCRASQSVSSAVAWYQQKPGKAPKLLIYSASSLYSGVPS |
| | | RFSGSRSGTDFTLTISSLQPEDFATYYCQQYWYGVAPITFGQGTK |
| | | VEIKRTVAAPSVFIFPPSDEQLKSGTASVVCLLNNFYPREAKVQW |
| | | KVDNALQSGNSQESVTEQDSKDSTYSLSSTLTLSKADYEKHKVY |
| | | ACEVTHQGLSSPVTKSFNRGEC |
| SWAP3 | 7 | EVQLVESGGGLVQPGGSLRLSCAASGFTFTSYYISWVRQAPG |
| heavy | | KGLEWVAEISPYSGSTYYADSVKGRFTISADTSKNTAYLQMNS |
| chain | | LRAEDTAVYYCALRARPPIRLHPRGSVMDYWGQGTLVTVSS |
| (FIG. 6) | | GGGGSGGGGSGGGGSEVQLVESGGGLVQPGGSLRLSCAASGFNISY |
| | | SYIHWVRQAPGKGLEWVASIYSSSGSTSYADSVKGRFTISADTSKNTAY |
| | | LQMNSLRAEDTAVYYCARGAIDYWGQGTLVTVSSASTKGPSVFPLAPS |
| | | SKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCP |
| | | PCPAPEAAGGPSVFLFPPKPKDTLMISRTPEVTCVVVDVSHEDPEVK |
| | | FNWYVDGVEVHNAKTKPREEQYNSTYRVVSVLTVLHQDWLNGKEYK |
| | | CKVSNKALGAPIEKTISKAKGQPREPQVYTLPPSREEMTKNQVSL |
| | | TCLVKGFYPSDIAVEWESNGQPENNYKTTPPVLDSDGSFFLYS |
| | | KLTVDKSRWQQGNVFSCSVMHEALHNHYTQKSLSLSPGK |
| SWAP3 | 8 | DIQMTQSPSSLSASVGDRVTITCRASQDVSTAVAWYQQKPGKAPK |
| light chain | | LLIYSASFLYSGVPSRFSGSGSGTDFTLTISSLQPEDFATYYCQQSY |
| (FIG. 6) | | TTPPTFGQGTKVEIKGGGGSGGGGSGGGGSDIQMTQSPSSLSASV |
| | | GDRVTITCRASQSVSSAVAWYQQKPGKAPKLLIYSASSLYSGVPS |
| | | RFSGSRSGTDFTLTISSLQPEDFATYYCQQWYSSGHVLITFGQGTK |
| | | VEIKRTVAAPSVFIFPPSDEQLKSGTASVVCLLNNFYPREAKVQW |
| | | KVDNALQSGNSQESVTEQDSKDSTYSLSSTLTLSKADYEKHKVY |
| | | ACEVTHQGLSSPVTKSFNRGEC |
| SWAP4 | 9 | EVQLVESGGGLVQPGGSLRLSCAASGFTFTSYYISWVRQAPG |
| heavy | | KGLEWVAEISPYSGSTYYADSVKGRFTISADTSKNTAYLQMN |
| chain | | SLRAEDTAVYYCALRARPPIRLHPRGSVMDYWGQGTLVTVSS |
| (FIG. 6) | | GGGGSGGGGSGGGGSEVQLVESGGGLVQPGGSLRLSCAASGFNISY |
| | | YYIHWVRQAPGKGLEWLASIYPSSGYTYYADSVKGRFTISADTSKNTAY |
| | | LQMNSLRAEDTAVYYCARSSFYWAMDYWGQGTLVTVSSASTKGPSVF |
| | | PLAPSSKSTSGGTAALGCLVKDYFPEPVTVSWNSGALTSGVHTFPAVL |
| | | QSSGLYSLSSVVTVPSSSLGTQTYICNVNHKPSNTKVDKKVEPKSCDK |
| | | THTCPPCPAPEAAGGPSVFLFPPKPKDTLMISRTPEVTCVVVDVSHE |
| | | DPEVKFNWYVDGVEVHNAKTKPREEQYNSTYRVVSVLTVLHQDWLN |
| | | GKEYKCKVSNKALGAPIEKTISKAKGQPREPQVYTLPPSREEMTK |
| | | NQVSLTCLVKGFYPSDIAVEWESNGQPENNYKTTPPVLDSDG |
| | | SFFLYSKLTVDKSRWQQGNVFSCSVMHEALHNHYTQKSLSLS |
| | | PGK |
| SWAP4 | 10 | DIQMTQSPSSLSASVGDRVTITCRASQDVSTAVAWYQQKPGKAPK |
| light chain | | LLIYSASFLYSGVPSRFSGSGSGTDFTLTISSLQPEDFATYYCQQSY |
| (FIG. 6) | | TTPPTFGQGTKVEIKGGGGSGGGGSGGGGSDIQMTQSPSSLSASV |
| | | GDRVTITCRASQSVSSAVAWYQQKPGKAPKLLIYSASSLYSGVPS |
| | | RFSGSRSGTDFTLTISSLQPEDFATYYCQQSYAAYLFTFGQGTKV |
| | | EIKRTVAAPSVFIFPPSDEQLKSGTASVVCLLNNFYPREAKVQWK |
| | | VDNALQSGNSQESVTEQDSKDSTYSLSSTLTLSKADYEKHKVYA |
| | | CEVTHQGLSSPVTKSFNRGEC |
| SWAP6 | 11 | DVQLVESGGGLVQAGGSLRLACAGSGRIFAIYDIAWYRHPPGNQ |
| light chain | | RELVAMIRPVVTEIDYADSVKGRFTISRNNAMKTVYLQMNNLKP |
| (FIG. 6) | | EDTAVYYCNAKRPWGSRDEYWGQGTQVTVSSGSGSGSYVLTQPP |
| | | SVSVSPGQTASITCSGDKVGHKYASWYQQKPGQSPVLVIYEDSQ |
| | | RPSGIPVRFSGSNSGNTATLTISGTQAMDEADYYCQAWDSSTDV |
| | | VFGGGTKLTVLGQPKAAPSVTLFPPSSEELQANKATLVCLISDFY |
| | | PGAVTVAWKADSSPVKAGVETTTPSKQSNNKYAASSYLSLTPEQ |
| | | WKSHRSYSCQVTHEGSTVEKTVAPTECS |
| SWAP 6 | 12 | QVQLQQWGAGLLKPSETLSLTCAVSGASFSGHYWTWIRQPP |
| heavy | | GKGLEWIGEIDHTGSTNYEPSLRSRVTISVDTSKNQFSLNLKS |
| chain | | VTAADTAVYYCARGGQGGYDWGHYHGLDVWGQGTTVTVSS |
| (FIG. 6) | | ASTKGPSVFPLAPSSKSTSGGTAALGCLVKDYFPEPVTVSWNSGALTS |
| | | GVHTFPAVLQSSGLYSLSSVVTVPSSSLGTQTYICNVNHKPSNTKVDKK |
| | | VEPKSCDKTHTCPPCPAPEAAGGPSVFLFPPKPKDTLMISRTPEVTC |
| | | VVVDVSHEDPEVKFNWYVDGVEVHNAKTKPREEQYNSTYRVVSVLTV |
| | | LHQDWLNGKEYKCKVSNKALGAPIEKTISKAKGQPREPQVYTLPPS |
| | | REEMTKNQVSLTCLVKGFYPSDIAVEWESNGQPENNYKTTPP |
| | | VLDSDGSFFLYSKLTVDKSRWQQGNVFSCSVMHEALHNHYT |
| | | QKSLSLSPGK |

FZD heavy chain = bold
CH1 = italics
hinge = bold italics
CH2 = underline italics
CH3 = bold underline
LRP VHH = bold italic underline (attached to N term of FZD fab VL)
FZD VL = plain text (not underline and not bold and not italics)
FZD CL = underline (not bold and not italics) and at C-terminus
Linkers = underline and SSGSGSGS or GGGGSGGGGSGGGGS

### VI. Animal Model of Bleomycin-Induced Acute and Chronic Lung Fibrosis

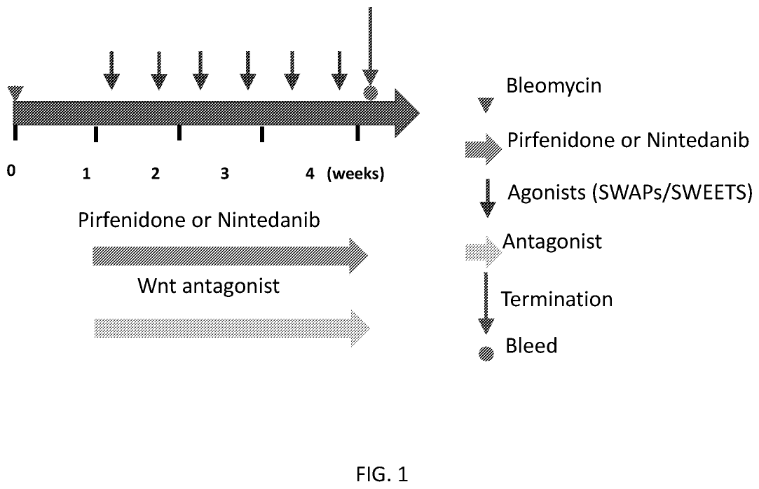

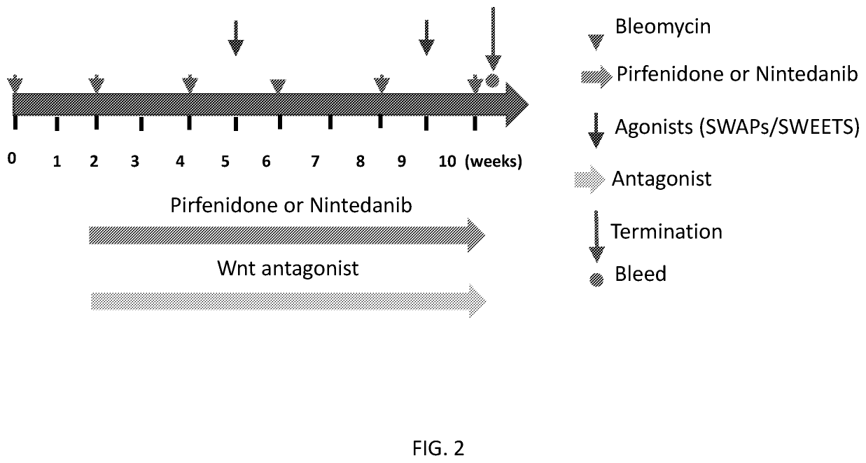

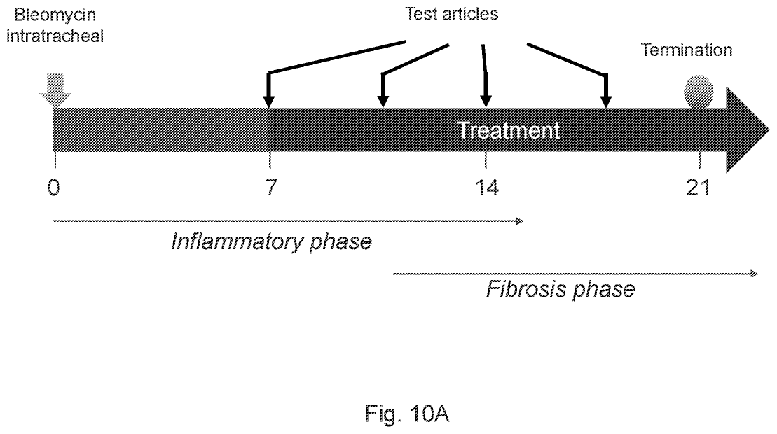

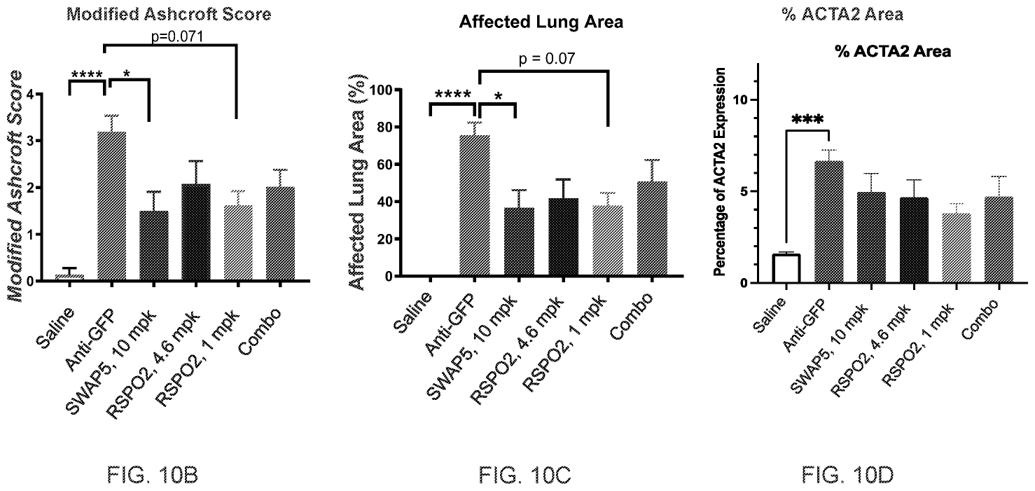

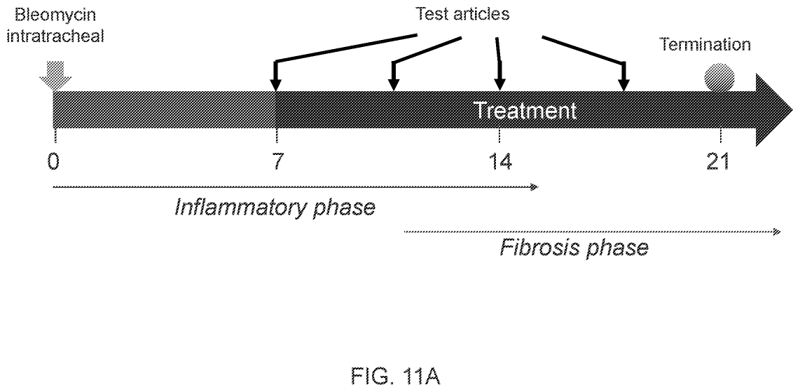

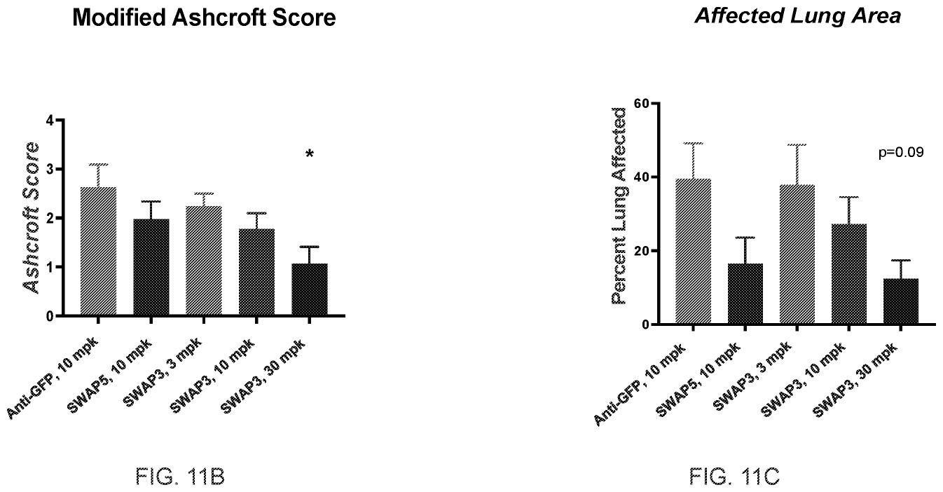

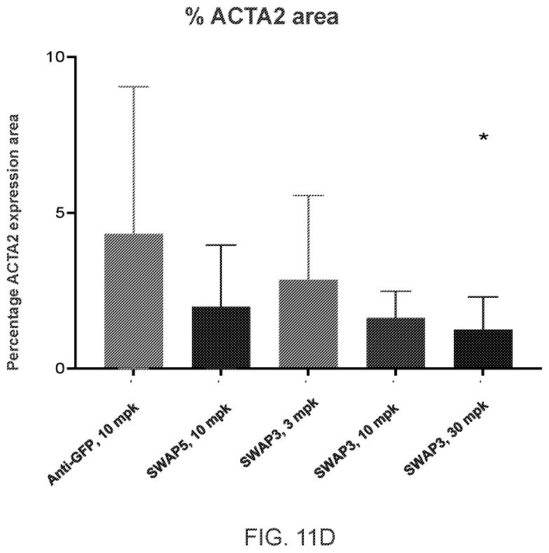

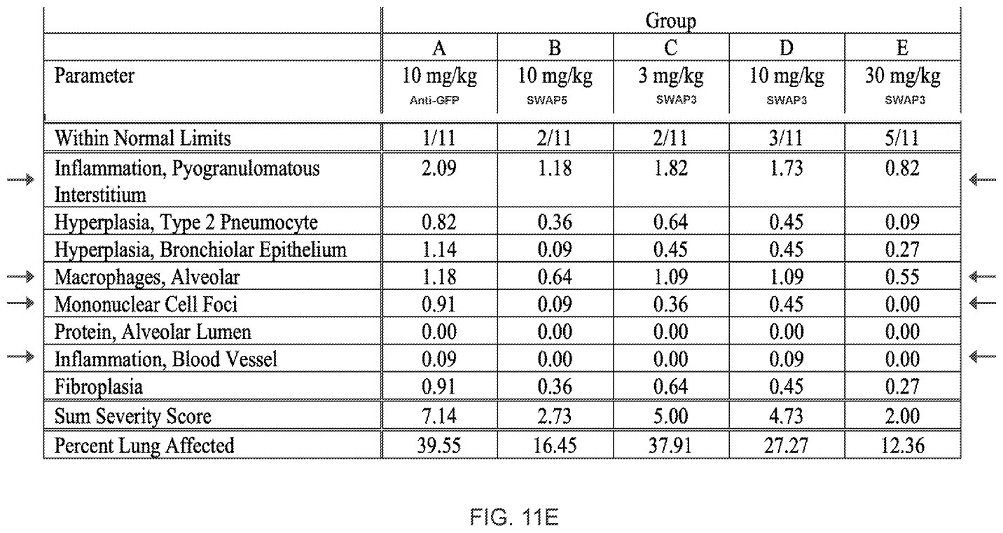

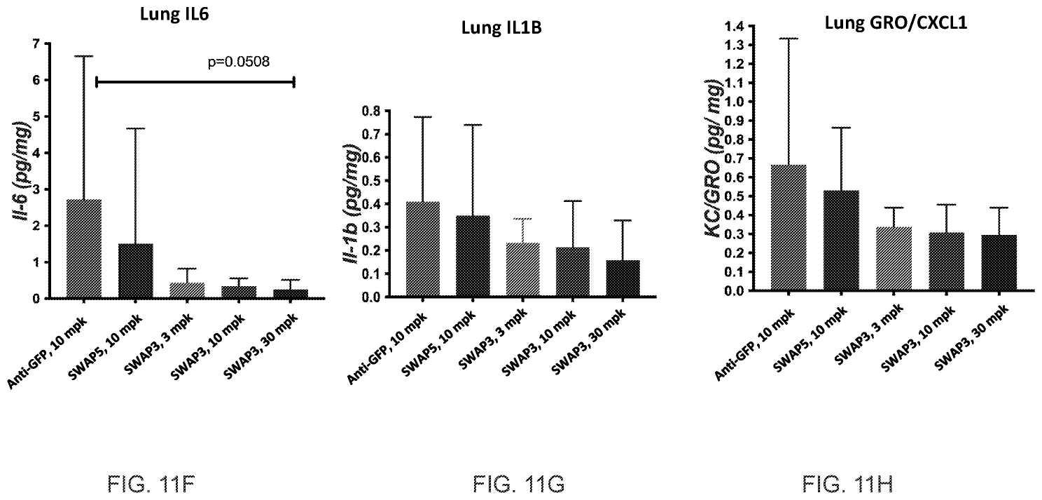

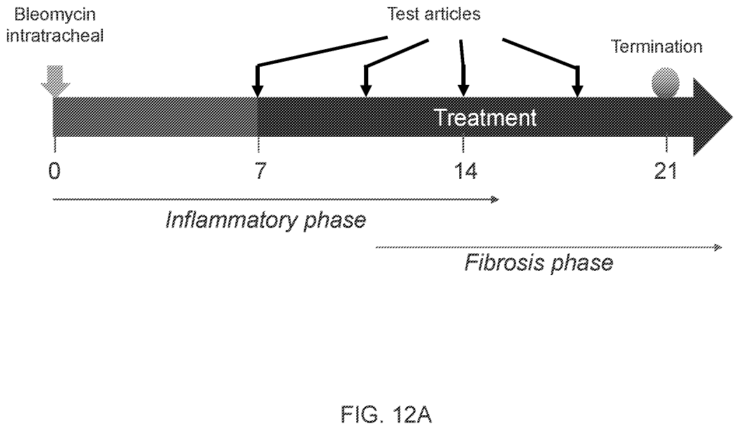

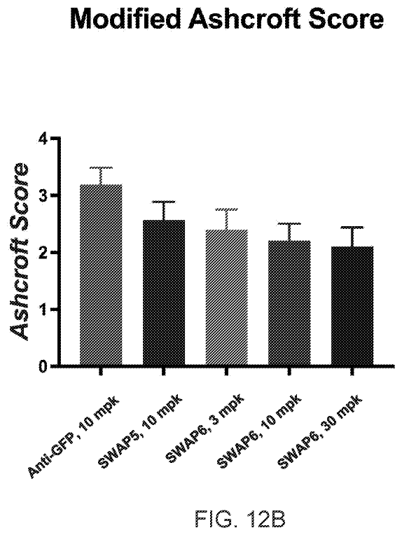

The acute lung injury (single bleomycin treatment; FIG. 1) and chronic intermittent/low dose bleomycin mouse models (FIG. 2) were used to determine the therapeutic effects of Wnt antagonist and agonist (administered concurrently or sequentially or individually) in combination with or without anti-fibrotic drugs (e.g., pirfenidone and nintedanib) to assess effects on anti-fibrotic and improved respiratory functions (see, e.g., Degryse, A. et al. (2010) Am J Physiol Lung Cell Mol Physiol. 299:442-52). Improvement of fibrosis was observed upon treatment with a WNT agonist, SWAP5 (see, e.g., FIG. 7, FIG. 10). Specifically, bleomycin was administered intratracheally to the mice at day 0, and animals were treated twice weekly with an agonist approach—combination of SWAP5 (FZD1,2,5,7,8 multi-FZD-specific SWAP) and RSPO2 (combo)—for two weeks starting at day 7 until termination at day 21. This led to a reduction in the degree of fibrosis severity and the area of the lung affected by fibrosis (FIG. 7). This demonstrated that Wnt signaling agonism can reduce pulmonary fibrosis. Improvement of fibrosis was also observed upon treatment with a Wnt agonist or the Wnt signaling enhancer RSPO2 alone (FIG. 10). For example, SWAP5 alone or RSPO2 alone reduced the fibrosis score and percentage of lung area affected (FIG. 10). A FZD5,8-specific SWAP3 alone reduced the fibrosis score, the percentage of lung affected, immune cell infiltration, and cytokine levels in the acute bleomycin mouse model in a dose-dependent manner (FIG. 11), and a FZD1,2,7-specific SWAP6 also caused a reduction in fibrosis (FIG. 12)

In these animal models, a Wnt/β-catenin signaling antagonist is applied, either general or cell-type specific, to reduce the proliferation of fibrogenic myofibroblasts and the production of extracellular matrix components contributing to fibrosis. Then either sequentially or concurrently or independently, an agonist that specifically targets the AT2/AEP cells to promote their self-renewal is applied or an agonist that targets the relevant immune cells such as macrophages to promote an anti-inflammatory state is applied. Upon withdrawal of that AT2-specific agonist, a subset of AT2 cells will spontaneously differentiate into new AT1 cells, instigated by the reduced level of Wnt/β-catenin signaling activity and/or the activity of the antagonist. In an additional study, a combination of antagonist targeted to fibrogenic myofibroblasts and an agonist targeting AT2/AEP cells applies, followed by successive application of an antagonist alone that targets both fibrogenic myofibroblasts and AT2 cells, reducing fibrosis and promoting AT2->ATI cell conversion. In an additional study, a macrophage specific Wnt agonist may be used to promote an anti-inflammatory state to facilitate tissue repair.

### VII. Animal Model of Emphysema

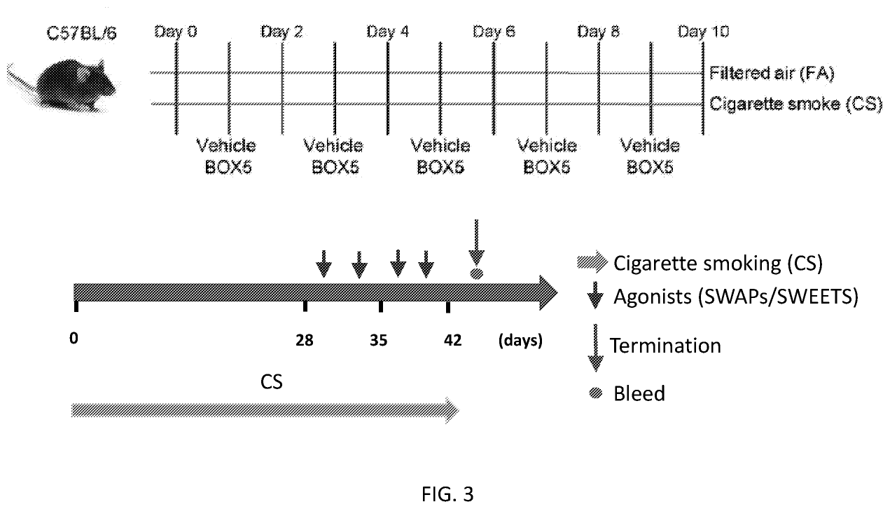

The cigarette smoking-induced model (FIG. 3) is used to establish emphysema in mice in order to evaluate the therapeutic effects of Wnt agonists (see above) in the regeneration of alveolar epithelial cells and improved respiratory functions (see, e.g., Baarsma, H. et al. (2017) J Exp Med. 214:143-163).

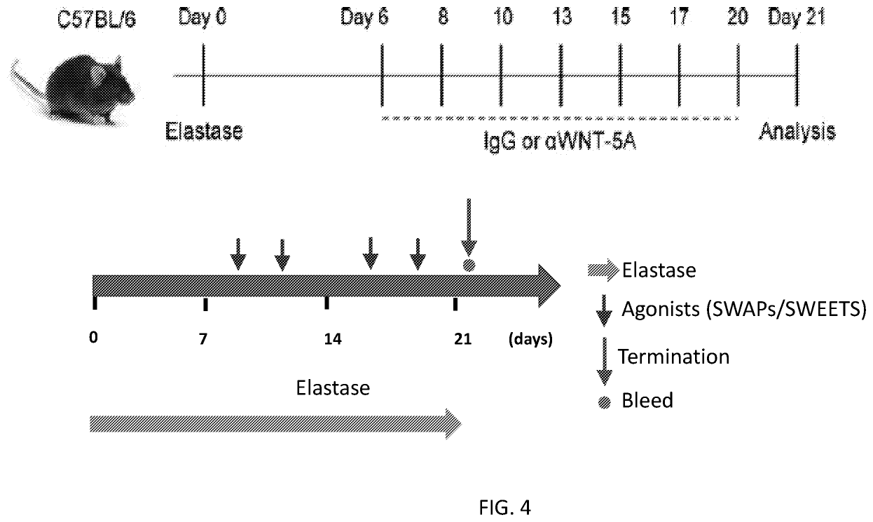

An elastase-induced emphysema mouse model (FIG. 4) is also used to establish the emphysema in mice and evaluate the therapeutic effects of Wnt agonist in the regeneration of alveolar epithelial cells and improved respiratory functions (see, e.g., Baarsma et al. supra).

Animals with induced emphysema are treated with pulsatile Wnt/β-catenin signaling agonism to promote AT2 cell proliferation followed by AT2->ATI conversion without coupling to antagonism to prevent fibrosis. In a second approach, animals are treated with a general or cell type-specific Wnt/β-catenin antagonist to limit fibrosis and then in sequence or concurrently a Wnt agonist is applied that specifically activates AT2 cells to proliferate and self-renew. This is followed by the removal of the AT2 specific Wnt agonist, thus allowing for AT2 differentiation into AT1 cells upon reduced Wnt signaling agonism and/or antagonist activity.

### REFERENCES

Adams, T. S., Schupp, J. C., Poli, S., Ayaub, E. A., Neumark, N., Ahangari, F., Chu, S. G., Raby, B. A., Deluliis, G., Januszyk, M., et al. (2020). Single-cell RNA-seq reveals ectopic and aberrant lung-resident cell populations in idiopathic pulmonary fibrosis. Sci Adv 6, eaba1983.
Akhmetshina, A., Palumbo, K., Dees, C., Bergmann, C., Venalis, P., Zerr, P., Horn, A., Kireva, T., Beyer, C., Zwerina, J., et al. (2012). Activation of canonical Wnt signalling is required for TGF-β-mediated fibrosis. Nature Communications 3.
Aran, D., Looney, A. P., Liu, L., Wu, E., Fong, V., Hsu, A., Chak, S., Naikawadi, R. P., Wolters, P. J., Abate, A. R., et al. (2019). Reference-based analysis of lung single-cell sequencing reveals a transitional profibrotic macrophage. Nature Immunology 20, 163-172.
Baarsma, H. A., and Königshoff, M. (2017). ‘WNT-er is coming’: WNT signalling in chronic lung diseases. Thorax 72, 746-759.
Barkauskas, C. E., Cronce, M. J., Rackley, C. R., Bowie, E. J., Keene, D. R., Stripp, B. R., Randell, S. H., Noble, P. W., and Hogan, B. L. M. (2013). Type 2 alveolar cells are stem cells in adult lung. Journal of Clinical Investigation 123, 3025-3036.
Cao, H., Wang, C., Chen, X., Hou, J., Xiang, Z., Shen, Y., and Han, X. (2018). Inhibition of Wnt/β-catenin signaling suppresses myofibroblast differentiation of lung resident mesenchymal stem cells and pulmonary fibrosis. Scientific Reports 8.
Cao, Z., Lis, R., Ginsberg, M., Chavez, D., Shido, K., Rabbany, S. Y., Fong, G.-H., Sakmar, T. P., Rafii, S., and Ding, B.-S. (2016). Targeting of the pulmonary capillary vascular niche promotes lung alveolar repair and ameliorates fibrosis. Nature Medicine 22, 154-162.
Carlier, F. M., Dupasquier, S., Ambroise, J., Detry, B., Lecocq, M., Biétry-Claudet, C., Boukala, Y., Gala, J.-L., Bouzin, C., Verleden, S. E., et al. (2020). Canonical WNT pathway is activated in the airway epithelium in chronic obstructive pulmonary disease. EBioMedicine 61, 103034.
Chen, X., Shi, C., Meng, X., Zhang, K., Li, X., Wang, C., Xiang, Z., Hu, K., and Han, X. (2016). Inhibition of Wnt/β-catenin signaling suppresses bleomycin-induced pulmonary fibrosis by attenuating the expression of TGF-β 1 and FGF-2. Experimental and Molecular Pathology 101, 22-30.
Chen, X., Shi, C., Cao, H., Chen, L., Hou, J., Xiang, Z., Hu, K., and Han, X. (2018). The hedgehog and Wnt/β-catenin system machinery mediate myofibroblast differentiation of LR-MSCs in pulmonary fibrogenesis. Cell Death & Disease 9.
Conlon, T. M., John-Schuster, G., Heide, D., Pfister, D., Lehmann, M., Hu, Y., Ertüz, Z., Lopez, M. A., Ansari, M., Strunz, M., et al. (2020). Inhibition of LT β R signalling activates WNT-induced regeneration in lung. Nature 588, 151-156.
Guo, L., Wang, T., Wu, Y., Yuan, Z., Dong, J., Li, X., An, J., Liao, Z., Zhang, X., Xu, D., et al. (2016). WNT/β-catenin signaling regulates cigarette smoke-induced airway inflammation via the PPAR 8/p38 pathway. Lab Invest 96, 218-229.
Haas, M., Gómez Vázquez, J. L., Sun, D. I., Tran, H. T., Brislinger, M., Tasca, A., Shomroni, O., Vleminckx, K., and Walentek, P. (2019). A N-Tp63 Mediates Wnt/β-Catenin-Induced Inhibition of Differentiation in Basal Stem Cells of Mucociliary Epithelia. Cell Reports 28, 3338-3352.e6.
Habermann, A. C., Gutierrez, A. J., Bui, L. T., Yahn, S. L., Winters, N. I., Calvi, C. L., Peter, L., Chung, M.-I., Taylor, C. J., Jetter, C., et al. (2020). Single-cell RNA sequencing reveals profibrotic roles of distinct epithelial and mesenchymal lineages in pulmonary fibrosis. Sci Adv 6, eaba1972.
Henderson, W. R., Chi, E. Y., Ye, X., Nguyen, C., Tien, Y., Zhou, B., Borok, Z., Knight, D. A., and Kahn, M. Inhibition of Wnt/β-catenin/CREB binding protein (CBP) signaling reverses pulmonary fibrosis. MEDICAL SCIENCES 6.
Jiang, Z., Lao, T., Qiu, W., Polverino, F., Gupta, K., Guo, F., Mancini, J. D., Naing, Z. Z. C., Cho, M. H., Castaldi, P. J., et al. (2016). A Chronic Obstructive Pulmonary Disease Susceptibility Gene, FAM13A, Regulates Protein Stability of β-Catenin. Am J Respir Crit Care Med 194, 185-197.
Kim, H.-T., Yin, W., Nakamichi, Y., Panza, P., Grohmann, B., Buettner, C., Guenther, S., Ruppert, C., Kobayashi, Y., Guenther, A., et al. (2019). WNT/RYK signaling restricts goblet cell differentiation during lung development and repair. Proc Natl Acad Sci USA 116, 25697-25706.
Kim, T. H., Kim, S.-H., Seo, J.-Y., Chung, H., Kwak, H. J., Lee, S.-K., Yoon, H. J., Shin, D. H., Park, S. S., and Sohn, J. W. (2011). Blockade of the Wnt/β-Catenin Pathway Attenuates Bleomycin-Induced Pulmonary Fibrosis. The Tohoku Journal of Experimental Medicine 223, 45-54.
Kneidinger, N., Yildirim, A.Ö., Callegari, J., Takenaka, S., Stein, M. M., Dumitrascu, R., Bohla, A., Bracke, K. R., Morty, R. E., Brusselle, G. G., et al. (2011). Activation of the WNT/β-Catenin Pathway Attenuates Experimental Emphysema. American Journal of Respiratory and Critical Care Medicine 183, 723-733.
Kobayashi, Y., Tata, A., Konkimalla, A., Katsura, H., Lee, R. F., Ou, J., Banovich, N. E., Kropski, J. A., and Tata, P. R. (2020). Persistence of a regeneration-associated, transitional alveolar epithelial cell state in pulmonary fibrosis. Nat Cell Biol 22, 934-946.
Königshoff, M., Balsara, N., Pfaff, E.-M., Kramer, M., Chrobak, I., Seeger, W., and Eickelberg, O. (2008). Functional Wnt Signaling Is Increased in Idiopathic Pulmonary Fibrosis. PLOS ONE 3, e2142.
Lam, A. P., Herazo-Maya, J. D., Sennello, J. A., Flozak, A. S., Russell, S., Mutlu, G. M., Budinger, G. R. S., DasGupta, R., Varga, J., Kaminski, N., et al. (2014). Wnt Coreceptor Lrp5 Is a Driver of Idiopathic Pulmonary Fibrosis. American Journal of Respiratory and Critical Care Medicine 190, 185-195.
Nabhan, A. N., Brownfield, D. G., Harbury, P. B., Krasnow, M. A., and Desai, T. J. (2018). Single-cell Wnt signaling niches maintain stemness of alveolar type 2 cells. Science 359, 1118-1123.
Reyfman, P. A., Walter, J. M., Joshi, N., Anekalla, K. R., McQuattie-Pimentel, A. C., Chiu, S., Fernandez, R., Akbarpour, M., Chen, C.-I., Ren, Z., et al. (2019). Single-Cell Transcriptomic Analysis of Human Lung Provides Insights into the Pathobiology of Pulmonary Fibrosis. American Journal of Respiratory and Critical Care Medicine 199, 1517-1536.
Schmid, A., Sailland, J., Novak, L., Baumlin, N., Fregien, N., and Salathe, M. (2017). Modulation of Wnt signaling is essential for the differentiation of ciliated epithelial cells in human airways. FEBS Lett 591, 3493-3506.
Shi, J., Li, F., Luo, M., Wei, J., and Liu, X. (2017). Distinct Roles of Wnt/β-Catenin Signaling in the Pathogenesis of Chronic Obstructive Pulmonary Disease and Idiopathic Pulmonary Fibrosis. Mediators of Inflammation 2017, 1-16.
Skronska-Wasek, W., Mutze, K., Baarsma, H. A., Bracke, K. R., Alsafadi, H. N., Lehmann, M., Costa, R., Stornaiuolo, M., Novellino, E., Brusselle, G. G., et al. (2017). Reduced Frizzled Receptor 4 Expression Prevents WNT/B-Catenin-driven Alveolar Lung Repair in Chronic Obstructive Pulmonary Disease. American Journal of Respiratory and Critical Care Medicine 196, 172-185.
Spanjer, A. I. R., Baarsma, H. A., Oostenbrink, L. M., Jansen, S. R., Kuipers, C. C., Lindner, M., Postma, D. S., Meurs, H., Heijink, I. H., Gosens, R., et al. (2016). TGF-β-induced profibrotic signaling is regulated in part by the WNT receptor Frizzled-8. The FASEB Journal 30, 1823-1835.
Strunz, M., Simon, L. M., Ansari, M., Kathiriya, J. J., Angelidis, I., Mayr, C. H., Tsidiridis, G., Lange, M., Mattner, L. F., Yee, M., et al. (2020). Alveolar regeneration through a Krt8+ transitional stem cell state that persists in human lung fibrosis. Nat Commun 11, 3559.
Ulsamer, A., Wei, Y., Kim, K. K., Tan, K., Wheeler, S., Xi, Y., Thies, R. S., and Chapman, H. A. (2012). Axin Pathway Activity Regulates in Vivo pY654-B-catenin Accumulation and Pulmonary Fibrosis. Journal of Biological Chemistry 287, 5164-5172.
Wang, R., Ahmed, J., Wang, G., Hassan, I., Strulovici-Barel, Y., Hackett, N. R., and Crystal, R. G. (2011). Down-Regulation of the Canonical Wnt B-Catenin Pathway in the Airway Epithelium of Healthy Smokers and Smokers with COPD. PLOS ONE 6, e14793.
Wang, X., Dai, W., Wang, Y., Gu, Q., Yang, D., and Zhang, M. (2015). Blocking the Wnt/β-Catenin Pathway by Lentivirus-Mediated Short Hairpin RNA Targeting B-Catenin Gene Suppresses Silica-Induced Lung Fibrosis in Mice. International Journal of Environmental Research and Public Health 12, 10739-10754.
Zacharias, W. J., Frank, D. B., Zepp, J. A., Morley, M. P., Alkhaleel, F. A., Kong, J., Zhou, S., Cantu, E., and Morrisey, E. E. (2018). Regeneration of the lung alveolus by an evolutionarily conserved epithelial progenitor. Nature 555, 251-255.
Zhang, M., Haughey, M., Wang, N.-Y., Blease, K., Kapoun, A. M., Couto, S., Belka, I., Hoey, T., Groza, M., Hartke, J., et al. (2020). Targeting the Wnt signaling pathway through R-spondin 3 identifies an anti-fibrosis treatment strategy for multiple organs. PLOS ONE 15, e0229445.
Zhou, B., Magana, L., Hong, Z., Huang, L. S., Chakraborty, S., Tsukasaki, Y., Huang, C., Wang, L., Di, A., Ganesh, B., et al. (2020). The angiocrine Rspondin3 instructs interstitial macrophage transition via metabolic-epigenetic reprogramming and resolves inflammatory injury. Nat Immunol 21, 1430-1443.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments. These and other changes can be made to the embodiments in light of the above-detailed description.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A method of treating a subject suffering from a pulmonary disorder, comprising administering to the subject an engineered Wnt antagonist and/or an engineered Wnt agonist.

2. The method of claim 1, wherein the Wnt antagonist and Wnt agonist are administered concurrently.

3. The method of claim 1, wherein the Wnt antagonist and Wnt agonist are administered sequentially.

4. The method of claim 3, wherein the Wnt agonist is administered before the Wnt antagonist.

5. The method of claim 3, wherein the Wnt antagonist is administered before the Wnt agonist.

6. The method of claim 1, wherein the pulmonary disorder is an interstitial lung disease.

7. The method of claim 6, wherein the interstitial lung disease is selected from the group consisting of idiopathic pulmonary fibrosis, cryptogenic organizing pneumonia, desquamative interstitial pneumonitis, nonspecific interstitial pneumonitis, hypersensitivity pneumonitis, acute interstitial pneumonitis, interstitial pneumonia, systemic sclerosis-associated pulmonary fibrosis, sarcoidosis, asbestosis-induced fibrosis, lung injury as the result of acute and chronic lung infections (e.g., viral, bacterial, fungal), pneumonia, aspiration injuries, sepsis, acute respiratory distress syndrome.

8. The method of claim 1, wherein the pulmonary disorder is a chronic obstructive pulmonary disease (COPD).

9. The method of claim 8, wherein the COPD is selected from the group consisting of chronic bronchitis, emphysema, and chronic asthma.

10. The method of claim 1, wherein the engineered Wnt antagonist is selected from the group consisting of an engineered polypeptide, an engineered antibody containing at least one epitope binding domain, a small molecule, an siRNA, and an antisense nucleic acid molecule.

11. The method of claim 10, wherein the engineered Wnt antagonist incorporates a tissue targeting molecule.

12. The method of claim 11, wherein the tissue targeting molecule is an antibody or fragment thereof that binds to a tissue specific cell surface antigen, optionally wherein the tissue is lung tissue.

13. The method of claim 1, wherein the engineered Wnt agonist is selected from the group consisting of an engineered polypeptide, an engineered antibody containing at least one epitope binding domain, and a small molecule.

14. The method of claim 13, wherein the engineered Wnt agonist incorporates a tissue targeting molecule, optionally wherein the tissue is lung tissue.

15. The method of claim 14, wherein the tissue targeting molecule is an antibody or antibody fragment that binds to a tissue specific cell surface antigen, optionally wherein the tissue is lung tissue.

16. The method of claim 1 wherein the disorder is pulmonary fibrosis, and the subject is treated with the Wnt antagonist.

17. The method of claim 1, wherein the disorder is pulmonary fibrosis, and the subject is treated with the Wnt agonist, R-spondin mimetic, or a combination of the Wnt agonist and a tissue-specific Wnt signaling enhancer or an R-spondin mimetic.

18. The method of claim 1, wherein the Wnt agonist and/or the Wnt antagonist are multi FZD-specific or mono FZD-specific.

19. The method of claim 1, wherein the Wnt agonist is specific to FZD5,8 or to FZD1,2,7, and the disorder is an interstitial lung disease such as pulmonary fibrosis and/or COPD.

20. The method of claim 1, wherein the Wnt agonist or the Wnt antagonist is FZD4-mono-specific or FZDS-mono-specific.

21. The method of claim 1, wherein the disorder is COPD, and the subject is treated with the Wnt agonist.
